(19) United States
(12) Patent Application Publication
(10) Pub. No.: US 2012/0046272 A1
(43) Pub. Date: Feb. 23, 2012

(54) Novel Therapeutic Compounds

(75) Inventor: Ramesh SESHA, Windsor, NJ (US)

(73) Assignee: GRUNENTHAL GMBH, Aachen (DE)

(21) Appl. No.: 13/213,672

(22) Filed: Aug. 19, 2011

Related U.S. Application Data
(60) Provisional application No. 61/401,996, filed on Aug. 23, 2010.

(51) Int. Cl. A61K 31/55; C07C 237/14; C07D 333/20; C07C 237/08; C07D 265/30; A61K 31/5375; A61K 31/16; A61K 31/381; C07C 237/52; C07C 271/64; A61K 31/27; C07D 491/06; C07D 211/22; A61K 31/451; C07C 229/28; A61K 31/215; C07C 229/08; A61K 31/221; C07C 69/96; A61K 31/265; C07D 489/02; A61K 31/485; C07C 217/62; A61K 31/138; A61P 25/16; A61P 25/28; A61P 29/00; A61P 25/24; A61P 1/00; A61P 35/00; A61P 25/04; A61P 25/00; C07C 237/04

(52) U.S. Cl. 514/215; 564/193; 564/191; 549/77; 564/196; 544/162; 514/237.5; 514/613; 514/626; 514/438; 564/157; 514/616; 560/159; 514/506; 540/581; 546/234; 514/331; 560/125; 514/529; 560/173; 514/551; 558/275; 558/273; 514/512; 546/44; 514/282; 546/225; 564/353; 514/651

(57) ABSTRACT

The present invention describes a series of therapeutically active compounds of formula I,

X—Y—Z (I)

that are useful for treating a disorder in a mammal. In the formula I, X and Z, which may be same or different, are independently selected from substituted or unsubstituted alkyl, substituted or unsubstituted alkenyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalkyl; and Y is a linker selected from —O—, —S—, —NH—, —(CH₂)ₙ—, —CO—, —CONR^{a}—, —NR^{a}CO—, —NR^{a}COO—, —COO—, —CONR^{a}CO—, —CONR^{a}COO— and —COOCOO—. The compounds are useful to treat neurodegenerative disorders, depression, Alzheimer's disease, cognitive disorders, motor disorders, Parkinson's disease, drug addiction, behavioral disorders, inflammatory disorders, stomach disorders, cancers, acute pain, chronic pain and recurrent pain.

### FIELD OF INVENTION

The invention describes number of novel therapeutically active compounds that are useful for treating a disorder in a mammal.

### BACKGROUND OF THE INVENTION

The invention had the object of finding novel compounds having valuable properties, in particular those, which can be used for the preparation of medicaments.

The compounds of this can be used to treat a number of disorders and they include neurodegenerative disorders, including central nervous system disorders such as depression, Alzheimer's disease, cognitive disorders, motor disorders such as Parkinson's disease, drug addiction, behavioral disorders and inflammatory disorders, stomach disorders, cancers, and also serve as analgesics in the treatment of acute, chronic or recurrent pain.

There are a number of classes of therapeutic compounds used for treating various disorders including pain. These include acetaminophen, NSAIDs such as naproxen, meloxicam etc, Selective serotonin reuptake inhibitor (SSRIs) like citalopram and fluoxetine etc, CINODS such as naproxcinod, OPIATES such as morphine, tramadol, tapentadol, oxycodone etc, Antiepileptic drugs such as gabapentin, pregabalin, NMDA (N-methyl-D-aspartic acid) receptor antagonists like memantine, Norepinephrine Reuptake inhibitors such as reboxetine and atomoxetine, and Serotonin Norepinephrine Reuptake inhibitors (SNRIs) such as duloxetine etc and Acetylcholinestearase inhibitors such as rivastigmine and donepezil etc.

Opioid or opioid agonists class of drugs include morphine, the archetypical opioid, and various others such as, for example, codeine, dihydrocodeine, hydrocodone, hydromorphone, levorphanol, meperidine, buprenorphine, fentanyl, fentanyl derivatives, dipipanone, heroin, tapentadol tramadol, etorphine, dihydroetorphine, butorphanol, methadone, diamorphine, oxycodone, oxymorphone, pethidine and propoxyphene, etc. Opioid agonists chemically interact with areas or binding sites of the central nervous system related to the perception of pain, to movement, mood and behavior, and to the regulation of neuroendocrinological functions. Opioid agonists exhibit pharmacological properties that provide a range of therapeutic uses for patients in addition to analgesic use. Opioid agonists have been prescribed for effective use as hypnotics, sedatives, anti-diarrheal, anti-spasmodic, and anti-tussives.

Similarly, GABA (g-aminobutyric acid) is one of the major inhibitory transmitters in the central nervous system of mammals. A number of antiepileptic compounds such as GABA analogues (also referred to as voltage gated calcium channel blockers or alpha 2-delta ligands) with considerable pharmaceutical activity have been synthesized in the art. GABA analogue cis-4-aminocrotonic acid (3) selectively activates a third class of GABA receptors in the mammalian CNS.

Serotonin Norepinephrine Reuptake inhibitors (SNRIs) are a class of antidepressant used in the treatment of clinical depression and other affective disorders. They are also sometimes used to treat anxiety disorders, obsessive-compulsive disorder, attention deficit hyperactivity disorder (ADHD) and chronic neuropathic pain. They act upon two neurotransmitters in the brain that are known to play an important part in mood, namely, serotonin and norepinephrine. Examples of SNRIs include venlafaxine, duloxetine, milnacipran and desvenlafaxine etc.

Another class of therapeutic targets includes NMDA (N-methyl-D-aspartic acid) Receptors. The NMDA antagonists inhibit the action of the N-methyl D-aspartate receptor (NMDAR) and they include ketamine (K), dextromethorphan (DXM), phencyclidine (PCP) often used as analgesic agents.

Acetylcholinesterase is one of the most crucial enzymes for nerve response and function. Acetylcholinesterase (AChE) catalyzes the hydrolysis of acylcholinesters with a relative specificity for acetylcholine and is used for developing drugs to treat neurodegerative disorders.

Norepinephrine Reuptake inhibitors (NRI, NERI) or adrenergic reuptake inhibitor (ARI), is a type of drug which acts as a reuptake inhibitor for the neurotransmitters norepinephrine (noradrenaline) and epinephrine (adrenaline) by blocking the action of the norepinephrine transporter. They include Atomoxetine/Tomoxetine (Strattera), Mazindol (Mazanor, Sanorex), Reboxetine (Edronax, Vestra) and Viloxazine (Vivalan).

However, all these classes of drugs have one or more side effects that limit their use in treating a disorder for example—particularly over a long period of time. There is an unmet need to invent and develop better and novel chemical compounds that target one or more of this class of therapeutic targets.

The present inventor while working on novel therapeutic molecules invented a number of compounds that provide therapeutic benefits.

### SUMMARY OF THE INVENTION

The present invention provides novel compounds that have been found to possess therapeutic activity. Accordingly, compounds of the invention are expected to be useful and advantageous as therapeutic agents for those diseases and disorders that can be treated by modulation of number of receptors including but not limited to opiate, acetylcholinsterase, NMDA receptors, serotonin and/or norepinephrine reuptake.

One aspect of the invention relates to compound of formula (I):

X—Y—Z (I)

or pharmaceutically acceptable salt thereof; wherein,

X and Z, which may be same or different, are independently selected from substituted or unsubstituted alkyl, substituted or unsubstituted alkenyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalkyl;

Y is a linker selected from —O—, —S—, —NH—, —(CH₂)ₙ—, —CO—, —CONR^{a}—, —NR^{a}CO—, —NR^{a}COO—, —COO—, —CONR^{a}CO—, —CONR^{a}COO— and —COOCOO—,

at each occurrence, R^{a} is selected from hydrogen, substituted or unsubstituted alkyl, substituted or unsubstituted alkenyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalkyl; and

‘n’ is an integer selected from 0 to 8, both inclusive.

According to one embodiment, the present invention relates to compounds of the formula (Ia)

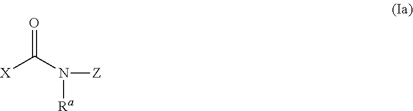

or pharmaceutically acceptable salt thereof;

wherein,

R^{a} is selected from hydrogen and substituted or unsubstituted alkyl; and

X and Z, which may be same or different, are independently selected from the group consisting of

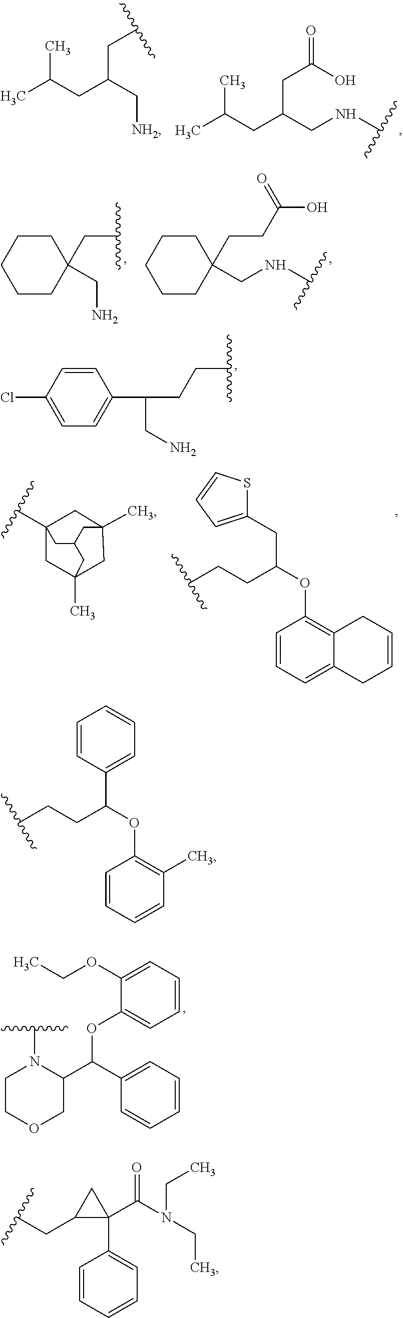

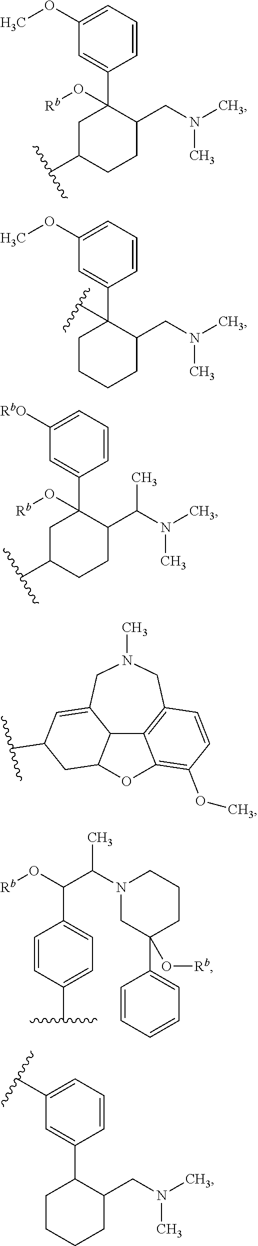

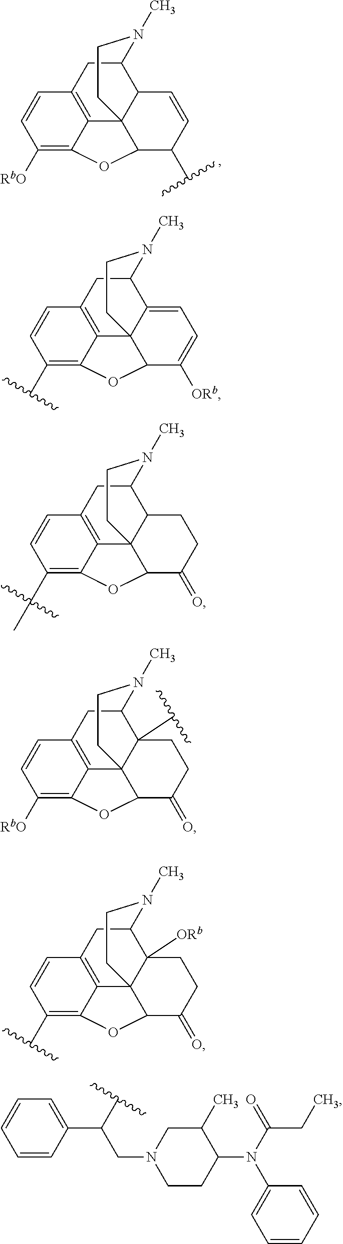

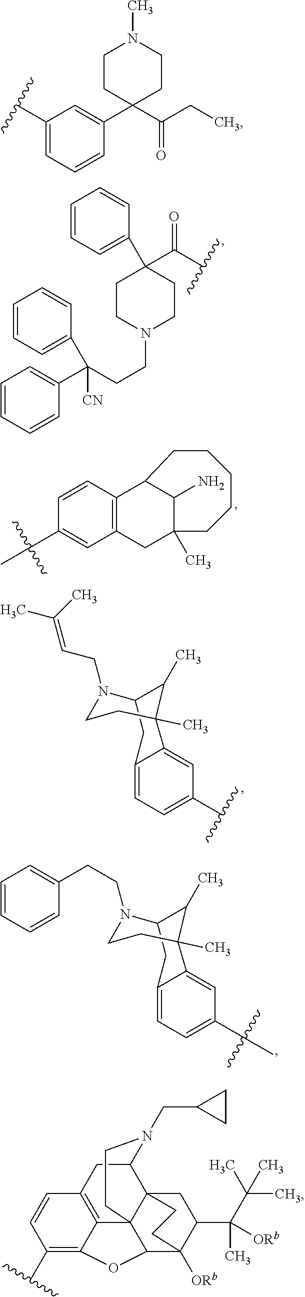

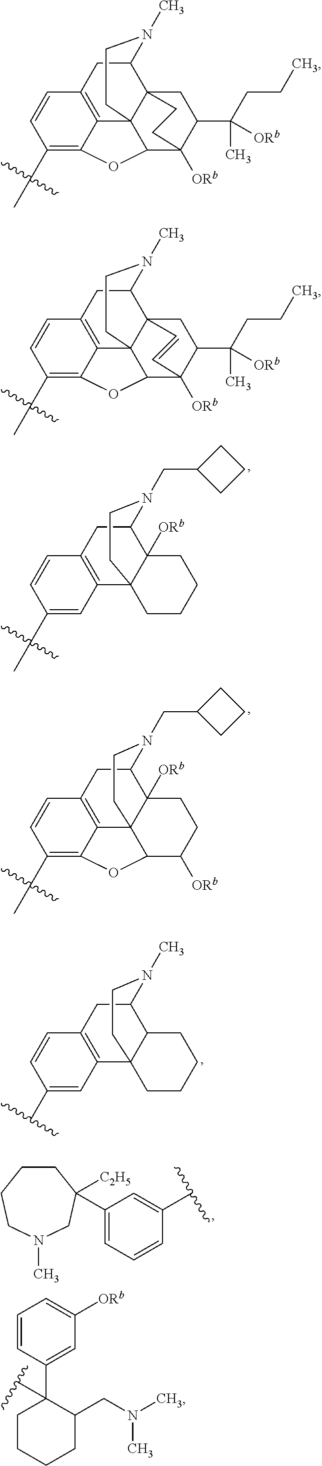

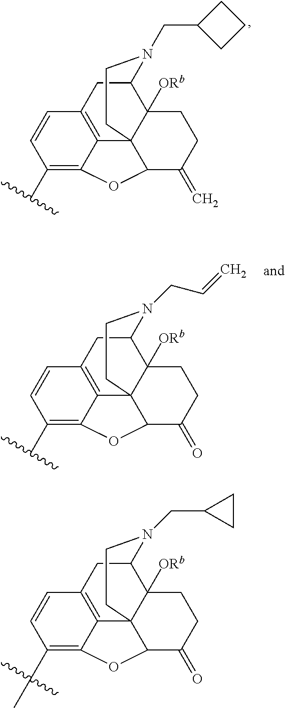

at each occurrence, R^{b} is selected from hydrogen, substituted or unsubstituted alkyl, substituted or unsubstituted alkenyl, substituted or unsubstituted cycloalkyl, substituted or unsubstituted cycloalkylalkyl, substituted or unsubstituted cycloalkenyl substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalky; or ^{b} is a protecting group.

According to another embodiment, the present invention relates to compounds of the formula (Ib)

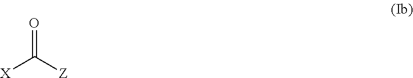

or pharmaceutically acceptable salt thereof;

wherein,

X and Z are as defined above in formula (Ia).

According to another embodiment, the present invention relates to compounds of the formula (Ic)

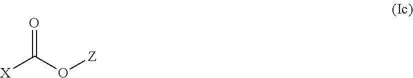

or pharmaceutically acceptable salt thereof;

wherein,

X and Z are as defined above in formula (Ia).

According to another embodiment, the present invention relates to compounds of the formula (Id)

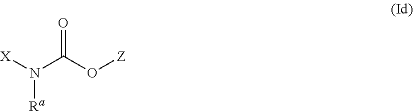

or pharmaceutically acceptable salt thereof;

wherein,

X, Z and R^{a} are as defined above in formula (Ia).

According to another embodiment, the present invention relates to compounds of the formula (Ie)

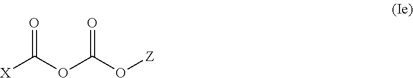

or pharmaceutically acceptable salt thereof;

wherein,

X and Z are as defined above in formula (Ia).

According to another embodiment, the present invention relates to compounds of the formula (If)

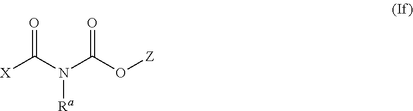

or pharmaceutically acceptable salt thereof;

wherein,

X, Z and R^{a} are as defined above in formula (Ia).

According to another embodiment, the present invention relates to compounds of the formula (Ig)

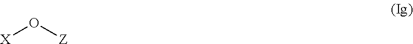

or pharmaceutically acceptable salt thereof;

wherein,

X and Z are as defined above in formula (Ia).

Certain compounds of formula (I), (Ia), (Ib), (Ic), (Id), (Ie), (If) and (Ig) are capable of existing in stereoisomeric forms (e.g. diastereomers and enantiomers). The present invention includes these stereoisomeric forms (including diastereomers and enantiomers) and mixtures of them.

The present invention also relates to pharmaceutical compositions comprising the compounds of the present invention and a pharmaceutically acceptable carrier.

The present invention also relates to methods for the treatment or prevention of disorders, diseases, or conditions in a mammal in need thereof by administering the compounds and pharmaceutical compositions of the present invention.

The present invention further relates to the use of the compounds of the present invention in the preparation of a medicament useful for the treatment or prevention of disorders, diseases, or conditions in a mammal in need thereof by administering the compounds and pharmaceutical compositions of the present invention.

Another aspect of the invention relates to a method of treating conditions comprising the step of administering a compound of formula (I), (Ia), (Ib), (Ic), (Id), (Ie), (If) and (Ig) wherein said condition is selected from the group consisting of psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Another aspect of the invention relates to a method of treating conditions comprising the step of administering a compound of formula (I), (Ia), (Ib), (Ic), (Id), (Ie), (If) and (Ig) wherein said condition is selected from the group consisting of schizophrenia, bipolar disorder, and obsessivecompulsive disorder.

### BRIEF DESCRIPTION OF FIGURES

FIG. 1 is a graph showing the time course of antinociceptive effect of compound of formula 31 against placebo in the thermal tail flick test.

FIGS. 2-6 are schematic diagrams of representative methods of manufacturing the compounds of this invention.

### DETAILED DESCRIPTION OF THE INVENTION

### Definitions

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl).

The term “alkenyl” refers to an aliphatic hydrocarbon group containing a carbon-carbon double bond and which may be a straight or branched chain having 2 to about 10 carbon atoms, e.g., ethenyl, 1-propenyl, 2-propenyl (allyl), iso-propenyl, 2-methyl-1-propenyl, 1-butenyl, and 2-butenyl.

The term “cycloalkyl” denotes a non-aromatic mono or multicyclic ring system of 3 to about 12 carbon atoms, such as cyclopropyl, cyclobutyl, cyclopentyl, and cyclohexyl. Examples of multicyclic cycloalkyl groups include, but are not limited to, perhydronapththyl, adamantyl and norbornyl groups, bridged cyclic groups or spirobicyclic groups, e.g., spiro(4,4) non-2-yl.

The term “cycloalkylalkyl” refers to a cyclic ring-containing radical having 3 to about 8 carbon atoms directly attached to an alkyl group. The cycloalkylalkyl group may be attached to the main structure at any carbon atom in the alkyl group that results in the creation of a stable structure. Non-limiting examples of such groups include cyclopropylmethyl, cyclobutylethyl, and cyclopentylethyl.

The term “cycloalkenyl” refers to a cyclic ring-containing radical having 3 to about 8 carbon atoms with at least one carbon-carbon double bond, such as cyclopropenyl, cyclobutenyl, and cyclopentenyl.

The term “aryl” means a carbocyclic aromatic system containing one, two or three rings wherein such rings may be fused. If the rings are fused, one of the rings must be fully unsaturated and the fused ring(s) may be fully saturated, partially unsaturated or fully unsaturated. The term “fused” means that a second ring is present (ie, attached or formed) by having two adjacent atoms in common (i.e., shared) with the first ring. The term “fused” is equivalent to the term “condensed”. The term “aryl” embraces aromatic radicals such as phenyl, naphthyl, tetrahydronaphthyl, indane and biphenyl.

The term “arylalkyl” refers to an aryl group as defined above directly bonded to an alkyl group as defined above, e.g., —CH₂C₆H₅ or —C₂H₅C₆H₅.

The term “heteroaryl” refers to an aromatic heterocyclic ring radical. The heteroaryl ring radical may be attached to the main structure at any heteroatom or carbon atom that results in the creation of a stable structure.

The term “heteroarylalkyl” refers to a heteroaryl ring radical directly bonded to an alkyl group. The heteroarylalkyl radical may be attached to the main structure at any carbon atom in the alkyl group that results in the creation of a stable structure.

The term “heterocyclyl” refers to a heterocyclic ring radical as defined above. The heterocyclyl ring radical may be attached to the main structure at any heteroatom or carbon atom that results in the creation of a stable structure.

The term “heterocyclylalkyl” refers to a heterocyclic ring radical directly bonded to an alkyl group. The heterocyclylalkyl radical may be attached to the main structure at any carbon atom in the alkyl group that results in the creation of a stable structure.

The terms “heterocyclic” and “heterocyclic ring” refer to a stable 3 to 15-membered ring radical which consists of carbon atoms and from one to five heteroatoms selected from nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heterocyclic or heteroaryl). Examples of such heterocyclic ring radicals include, but are not limited to, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazolyl, imidazolyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heterocyclic ring radical may be attached to the main structure at any heteroatom or carbon atom that results in the creation of a stable structure.

The term “dosage form” as used herein is defined to mean a solid oral pharmaceutical preparation or system in which doses of medicine or active drug are included. A dosage form will desirably comprise, for example, at least one slow release dosage form including various slow release forms such as, osmosis controlled-release dosage form, erosion controlled-release dosage form, dissolution controlled-release dosage form, diffusion controlled-release dosage form, controlled-release matrix core, controlled-release matrix core coated with at least one release-slowing coat, enteric coated dosage form, one sustained dosage, dosage form surrounded by at least one delayed-release coat, capsules, minitablets, caplets, uncoated microparticles, microparticles coated with release-slowing coat, microparticles coated with delayed-release coat or any combination thereof.

The term “effective amount” as used herein means a dosage which is sufficient in order for the treatment of the patient to be effective compared with no treatment.

The term “medicament” as used herein means a dosage form suitable for administration of the pharmaceutically active compound to a patient.

The term “optimal” us used herein means an amount which is the optimal dosage for that compound when used in single-compound therapy.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

The term “pain and pain related conditions” as used herein is defined as any pain due to a medical conditions including but not limited to neuropathic pain, osteoarthritis, rheumatoid arthritis, fibromyalgia, and back, musculoskeletal pain, Ankylosing spondylitis, juvenile rheumatoid arthritis, migraines, dental pain, abdominal pains, ischemic pain, postoperative pain or because of an anesthetic or surgical contrition.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder.

The term “therapeutically effective amount” means an amount that elicits a biological response in a mammal including the suboptimal amount.

The term “Pharmaceutically-acceptable salt” means a salt prepared by conventional means, and are well known by those skilled in the art. The pharmacologically acceptable salts” include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. When compounds of the invention include an acidic function such as a carboxy group, then suitable pharmaceutically acceptable cation pairs for the carboxy group are well known to those skilled in the art and include alkaline, alkaline earth, ammonium, quaternary ammonium cations and the like. For additional examples of “pharmacologically acceptable salts,” see infra and Berge et al., J. Pharm. Sci. 66:1 (1977).

The term “Saturated, partially-saturated or unsaturated” includes substituents saturated with hydrogens, substituents completely unsaturated with hydrogens and substituents partially saturated with hydrogens.

The term “Leaving group” generally refers to groups readily displaceable by a nucleophile, such as an amine, a thiol or an alcohol nucleophile. Such leaving groups are well known in the art. Examples of such leaving groups include, but are not limited to, N-hydroxysuccinimide, N-hydroxybenzotriazole, halides, triflates, tosylates and the like. Preferred leaving groups are indicated herein where appropriate.

The term “Protecting group” generally refers to groups well known in the art which are used to prevent selected reactive groups, such as carboxy, amino, hydroxy, mercapto and the like, from undergoing undesired reactions, such as nucleophilic, electrophilic, oxidation, reduction and the like. Preferred protecting groups are indicated herein where appropriate. Examples of amino protecting groups include, but are not limited to, aralkyl, substituted aralkyl, cycloalkenylalkyl and substituted cycloalkenyl alkyl, allyl, substituted allyl, acyl, alkoxycarbonyl, aralkoxycarbonyl, silyl and the like. Examples of aralkyl include, but are not limited to, benzyl, ortho-methylbenzyl, trityl and benzhydryl, which can be optionally substituted with halogen, alkyl, alkoxy, hydroxy, nitro, acylamino, acyl and the like, and salts, such as phosphonium and ammonium salts. Examples of aryl groups include phenyl, naphthyl, indanyl, anthracenyl, 9-(9-phenylfluorenyl), phenanthrenyl, durenyl and the like. Examples of cycloalkenylalkyl or substituted cycloalkylenylalkyl radicals, preferably have 6-10 carbon atoms, include, but are not limited to, cyclohexenyl methyl and the like. Suitable acyl, alkoxycarbonyl and aralkoxycarbonyl groups include benzyloxycarbonyl, t-butoxycarbonyl, iso-butoxycarbonyl, benzoyl, substituted benzoyl, butyryl, acetyl, trifluoroacetyl, trichloro acetyl, phthaloyl and the like. A mixture of protecting groups can be used to protect the same amino group, such as a primary amino group can be protected by both an aralkyl group and an aralkoxycarbonyl group. Amino protecting groups can also form a heterocyclic ring with the nitrogen to which they are attached, for example, 1,2-bis(methylene)benzene, phthalimidyl, succinimidyl, maleimidyl and the like and where these heterocyclic groups can further include adjoining aryl and cycloalkyl rings. In addition, the heterocyclic groups can be mono-, di- or tri-substituted, such as nitrophthalimidyl. Amino groups may also be protected against undesired reactions, such as oxidation, through the formation of an addition salt, such as hydrochloride, toluenesulfonic acid, trifluoroacetic acid and the like. Many of the amino protecting groups are also suitable for protecting carboxy, hydroxy and mercapto groups. For example, aralkyl groups. Alkyl groups are also suitable groups for protecting hydroxy and mercapto groups, such as tert-butyl.

The term “Silyl protecting groups” are silicon atoms optionally substituted by one or more alkyl, aryl and aralkyl groups. Suitable silyl protecting groups include, but are not limited to, trimethylsilyl, triethylsilyl, triisopropylsilyl, tert-butyldimethylsilyl, dimethylphenylsilyl, 1,2-bis(dimethylsilyl)benzene, 1,2-bis(dimethylsilyl)ethane and diphenylmethylsilyl. Silylation of an amino group provide mono- or di-silylamino groups. Silylation of aminoalcohol compounds can lead to a N,N,O-trisilyl derivative. Removal of the silyl function from a silyl ether function is readily accomplished by treatment with, for example, a metal hydroxide or ammonium fluoride reagent, either as a discrete reaction step or in situ during a reaction with the alcohol group. Suitable silylating agents are, for example, trimethylsilyl chloride, tert-butyl-dimethylsilyl chloride, phenyldimethylsilyl chloride, diphenylmethyl silyl chloride or their combination products with imidazole or DMF. Methods for silylation of amines and removal of silyl protecting groups are well known to those skilled in the art. Methods of preparation of these amine derivatives from corresponding amino acids, amino acid amides or amino acid esters are also well known to those skilled in the art of organic chemistry including amino acid/amino acid ester or aminoalcohol chemistry.

### General Methods of Preparation

The compounds described herein may be prepared by techniques known in the art. In addition, the compounds described herein may be prepared by following the reaction sequence as depicted in Schemes I to V. The starting materials are either commercially available or can be prepared by the procedures known in the art. Further, in the schemes, where specific bases, acids, reagents, solvents, coupling agents, etc., are mentioned, it is understood that other bases, acids, reagents, solvents, coupling agents etc., known in the art may also be used and are therefore included within the present invention. Variations in reaction conditions, for example, temperature and/or duration of the reaction, which may be used as known in the art are also within the scope of the present invention. All the isomers of the compounds described in these schemes, unless otherwise specified, are also encompassed within the scope of this invention.

The compounds of this invention have been prepared using a number of starting materials outlined in Formula Ito XXXIX.

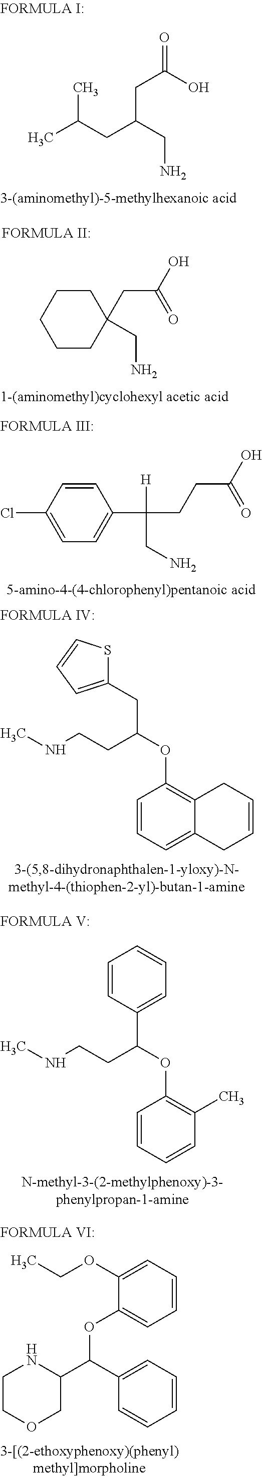

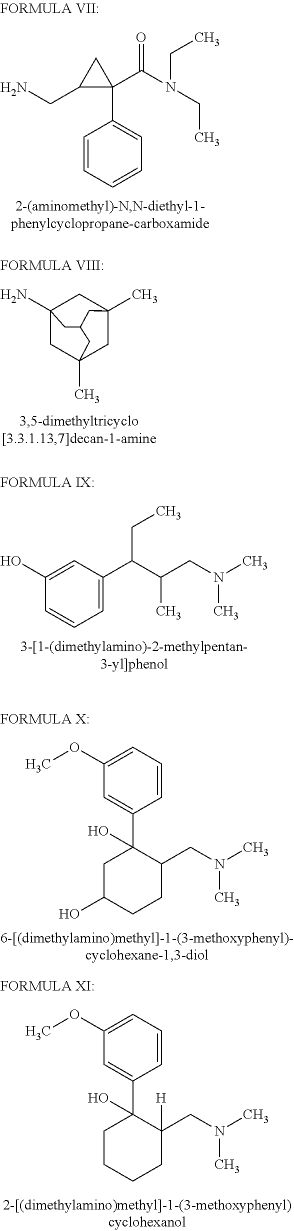

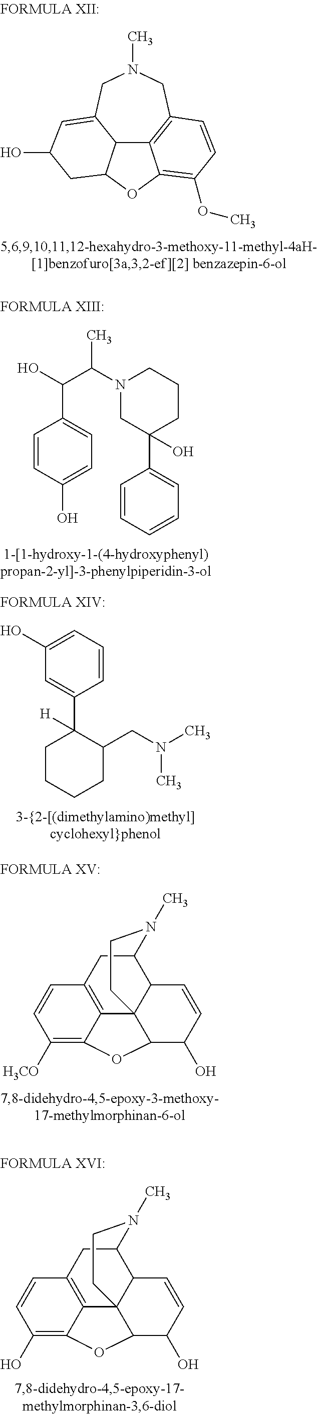

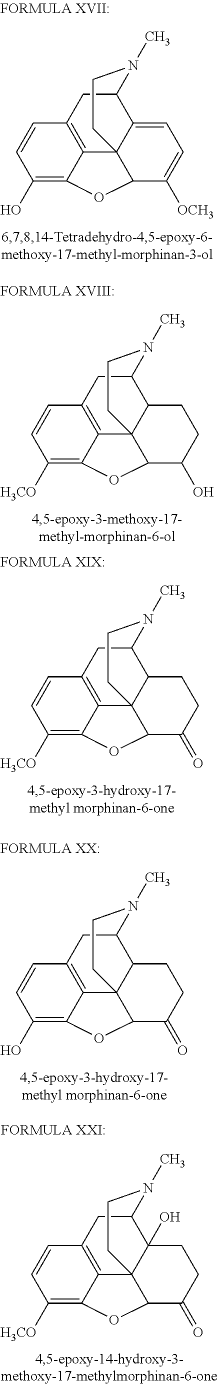

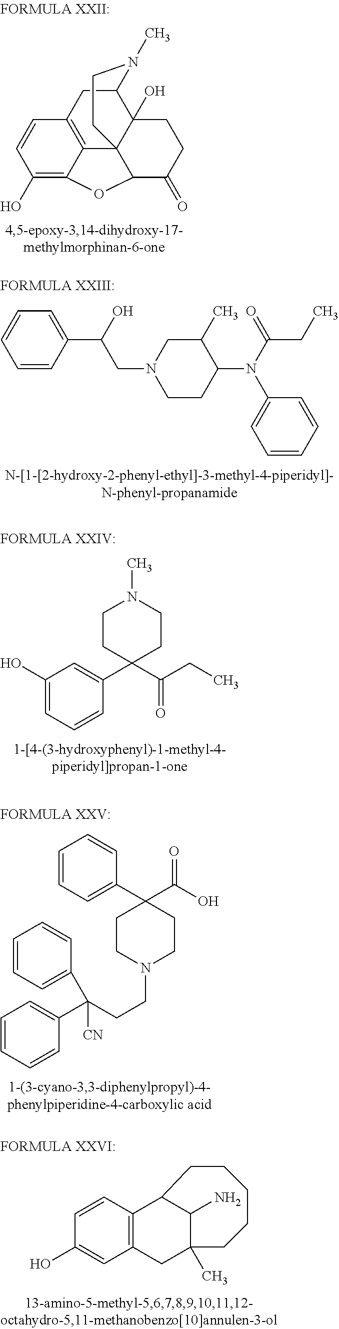

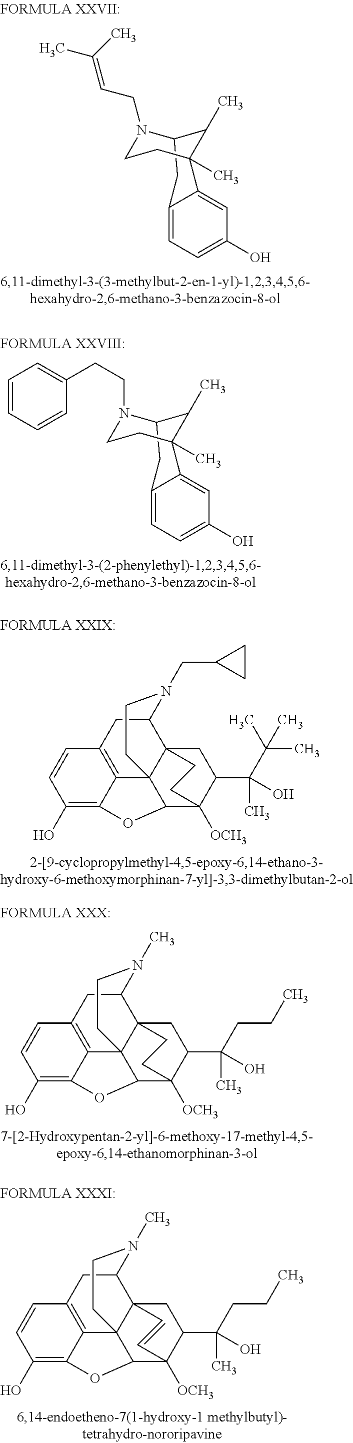

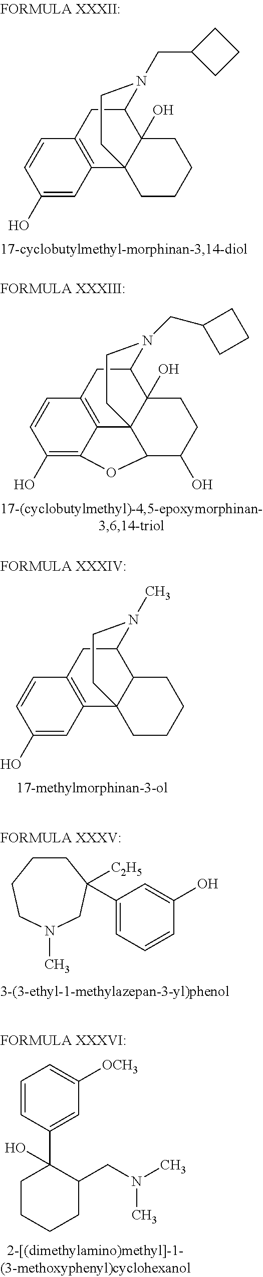

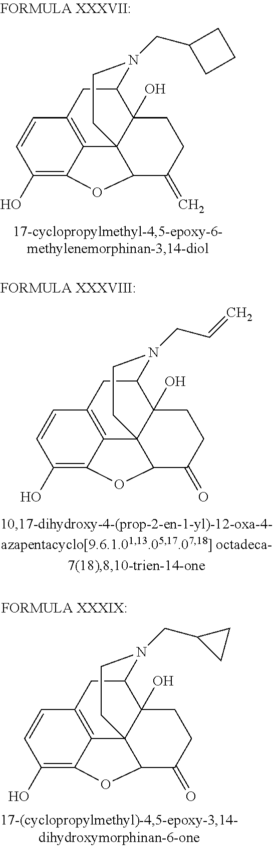

The starting materials were converted a variety of reactive starting materials such acid chlorides or protected starting material according to standard procedures known to the skilled in the art. Many procedures are available for forming amide bonds between an amine derivative formula (IV to VIII) and a carboxylic acid chloride (I to III) with the use of coupling agents. For Example the Amide formation was carried out according to the Scheme I. Procedures have been developed which use reagents such as carbodiimides as amide coupling agents. These carbodiimides include for example dicyclohexylcarbodiimide (DCC), diisopropylcarbodiimide (DIC), 1-(3-dimethylaminopropyl)-3-ethylcarbodiimide hydrochloride (EDC) and the like. Other amide coupling agents known in the art such as 1-ethoxycarbonyl-2-dihydroquinoline (EEDQ), phosphonium (e.g. phosphonium hexafluorophosphate (BOP), and others) or uronium-based reagents (e.g. TBTU, HATU and others) may also be used to form the amide bonds. In addition, anhydrides may also be utilized to form the desired amide bond. Catalysts such as 1-hydroxybenzotriazole (HOBT) and derivatives thereof have also been used. A summary of such methods is found in “Comprehensive Organic Transformations”, R. C. Larock, VCH Publishers (1989) pp. 972-972. An overview of such transformations is also available in “March's Advanced Organic Chemistry: Reactions, Mechanisms, and Structure (Sixth Edition)”, Michael B. Smith and Jerry March, Wiley-Interscience Publishers, (2007), pp 1431-1434.

Another general reaction for the preparation of amides is the treatment of acyl halides with amine. Such a transformation is well known to those skilled in the art and an overview of such transformations is available in “March's Advanced Organic Chemistry: Reactions, Mechanisms, and Structure (Sixth Edition)”, Michael B. Smith and Jerry March, Wiley-Interscience Publishers, (2007), pp. 1427-1429

Esterification was another mode of preparing the novel chemical entities as per this invention. A typical preparation was prepared according to Scheme II.

Similarly the carbamate compounds (Example 11 to 20) were prepared using an efficient solvent-free methodology in high yield and purity from relevant starting compounds, sodium cyanate and silica sulfuric acid. The typical Carbamate reaction was carried according to the Scheme III.

Etherification was another mode of preparing the novel chemical entities as per this invention. A typical preparation was prepared according to Scheme V. The free hydroxyl group of the compounds can be treated with suitable reagent to form ethers by procedures known in the art, for example the alcohol or phenol can be converted into corresponding alkoxide or aroxide, followed by reaction with alkyl halide or aryl halide. An overview of such transformations is also available in “March's Advanced Organic Chemistry: Reactions, Mechanisms, and Structure (Fourth Edition)”, Michael B. Smith and Jerry March, Wiley-Interscience Publishers, (2005), pp 386-387.

In all these reactions, protecting groups were employed to protected desired amino or carboxyl groups. The method of protecting desired groups are known to person skilled in the art.

A number of protecting groups are used to protect amine or carboxyl group that needs to be retained in the final compound. Protecting groups are removed under conditions which will not affect the remaining portion of the molecule. These methods are well known in the art and include acid hydrolysis, hydrogenolysis and the like. A preferred method involves removal of a protecting group, such as removal of a benzyloxycarbonyl group by hydrogenolysis utilizing palladium on carbon in a suitable solvent system such as an alcohol, acetic acid, and the like or mixtures thereof. A t-butoxycarbonyl protecting group can be removed utilizing an inorganic or organic acid, such as HCl or trifluoroacetic acid, in a suitable solvent system, such as dioxane or methylene chloride. The resulting amino salt can readily be neutralized to yield the free amine. Carboxy protecting group, such as methyl, ethyl, benzyl, tert-butyl, 4-methoxyphenylmethyl and the like, can be removed under hydrolysis and hydrogenolysis conditions well known to those skilled in the art.

The invention further contemplates separating the enantiomers in whole or in part of the present invention or synthesizing enantiomerically enriched compounds of the invention. The composition may be prepared by separating the enantiomers in whole or in part by standard methods, for example by chemical resolution using optically active acid or by use of column chromatography or reverse-phase column chromatography using a substantially optically active (or “chiral”) stationary phase as known to those skilled in the art. The formation and/or isolation of specific enantiomers of a compound is not routine, and there are no general methods that may be used to obtain specific enantiomers of all compounds. The methods and conditions used to obtain specific enantiomers of a compound must be determined for each specific compound.

Enantiomerically enriched compounds of the invention can also be obtained from enantiomerically enriched precursors.

Below are the representative compounds, which are illustrative in nature only and are not intended to limit to the scope of the invention.

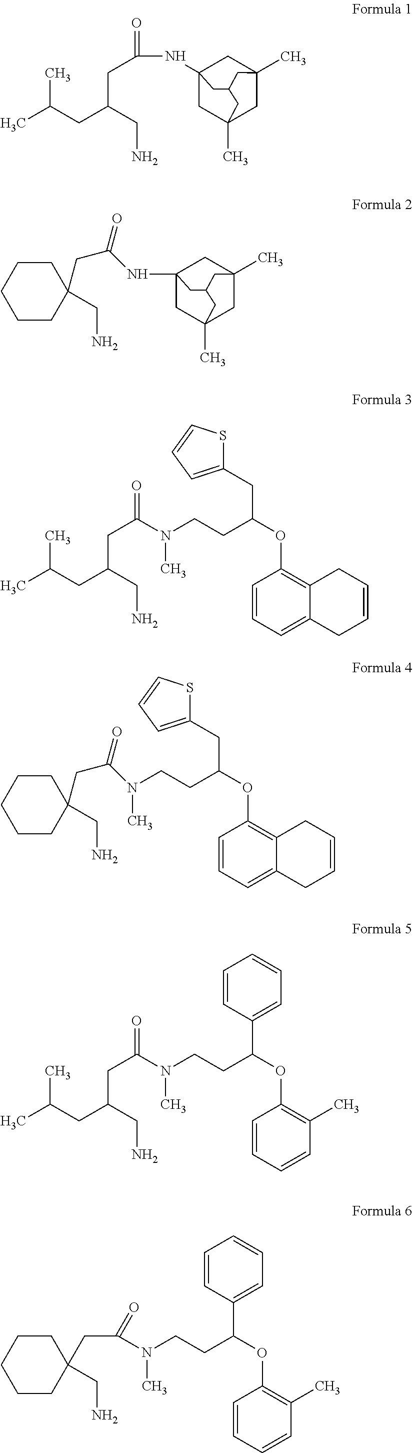

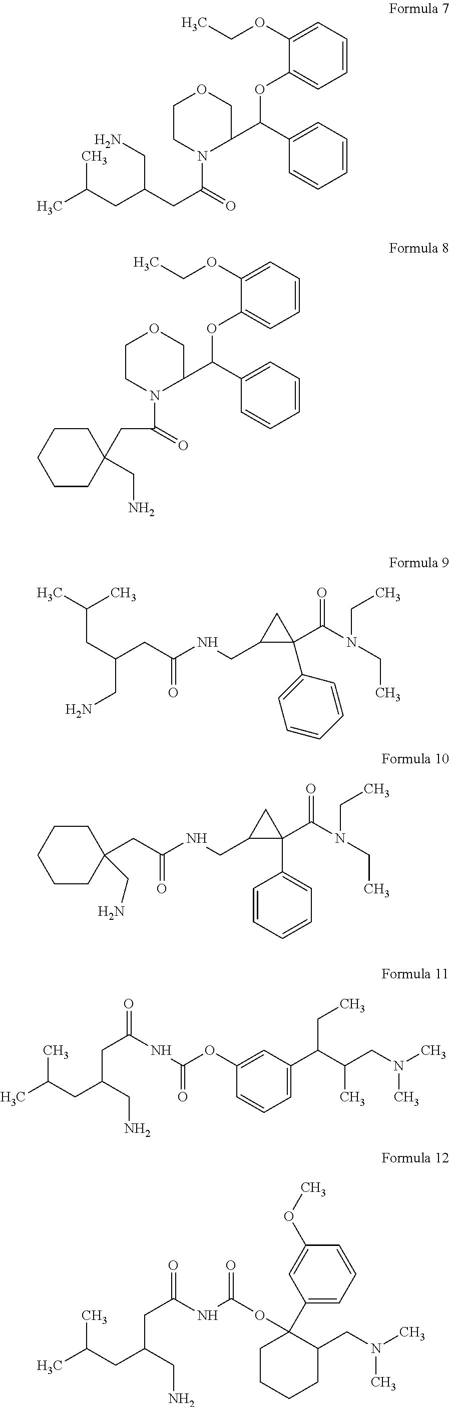

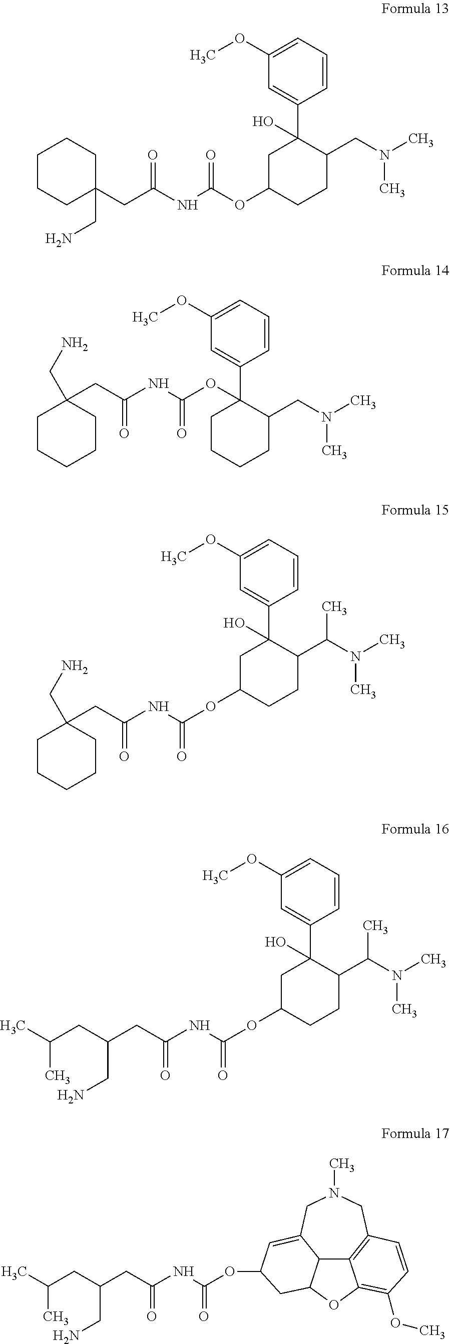

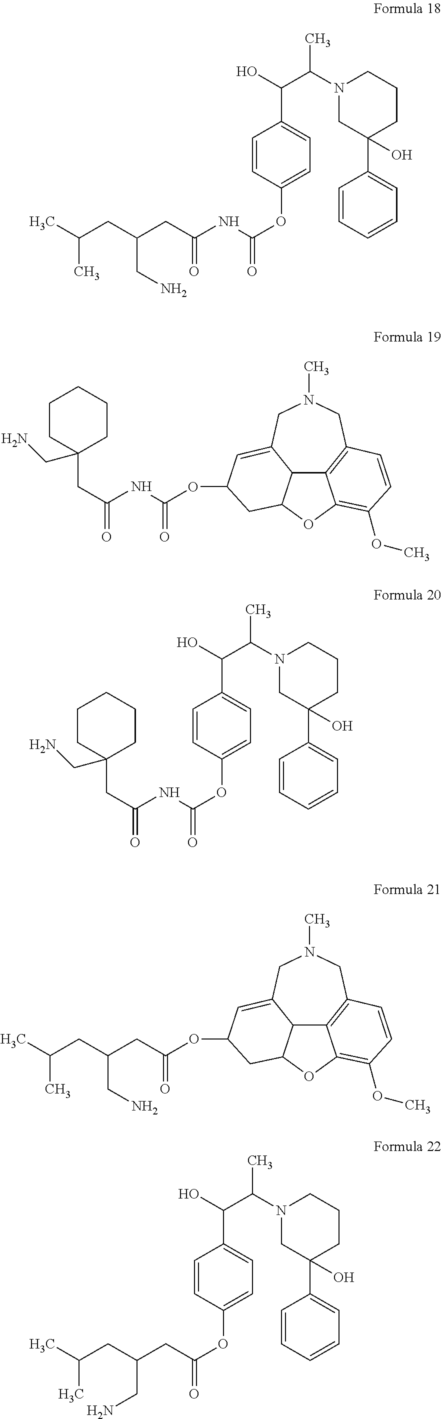

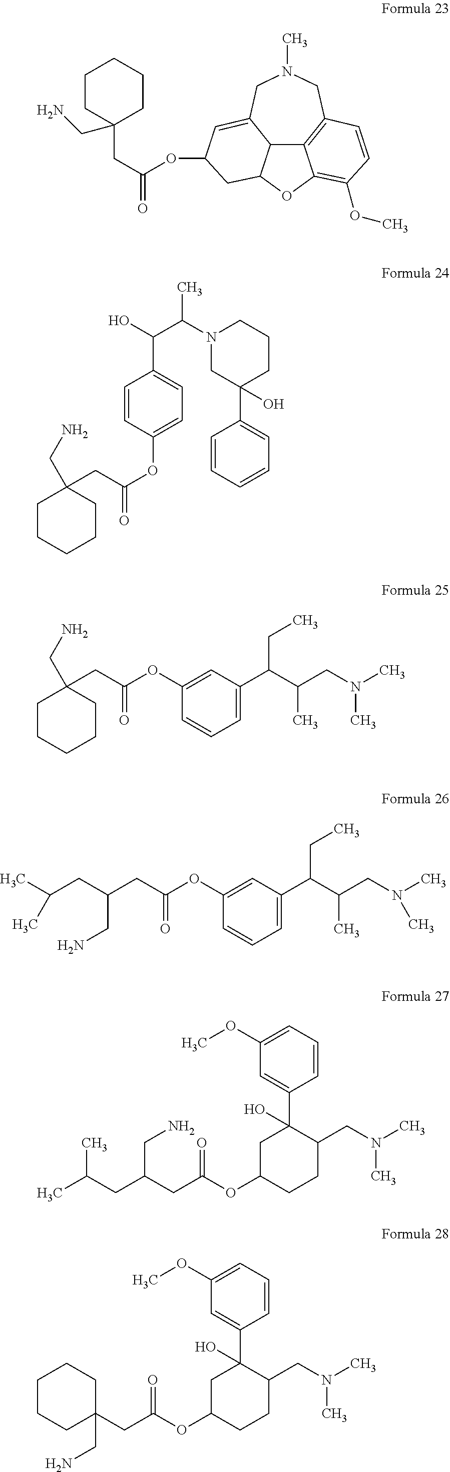

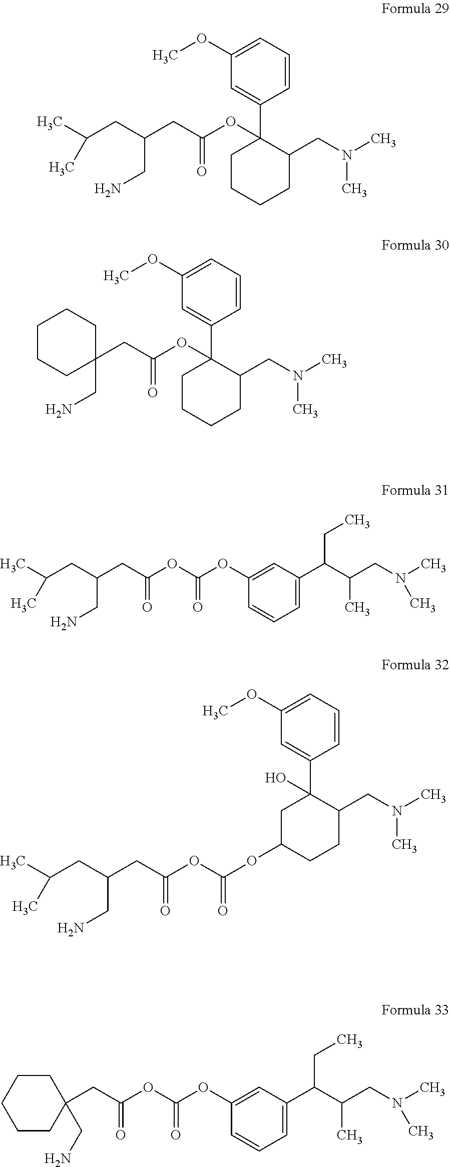

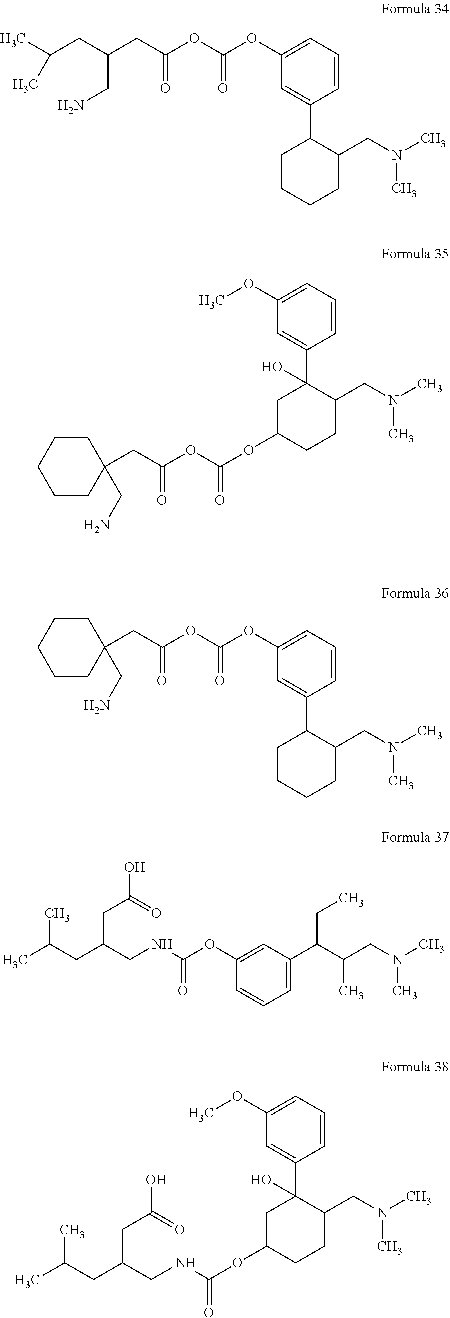

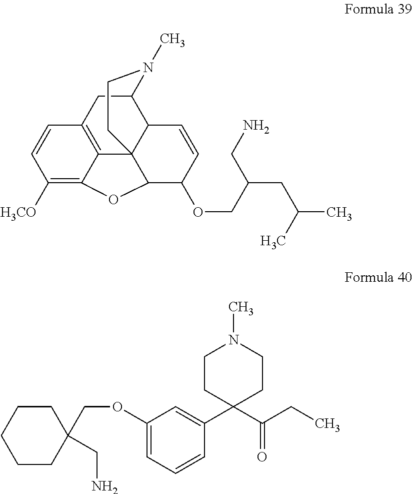

The following Examples are provided for illustrations and are in no way limit the scope of this invention.

### Example 1

### Preparation of Compound of Formula 1

The starting materials 3-(aminomethyl)-5-methylhexanoic acid (FORMULA I) and 3,5-dimethyltricyclo[3.3.1.13,7]decan-1-amine (FORMULA VIII) purified by standard recrystallization with Isopropyl Alcohol and Ethanol respectively. 3-(aminomethyl)-5-methylhexanoic acid (FORMULA I) as converted into its acid chloride, 3-(aminomethyl)-5-methylhexanoyl chloride either using Thionyl Chloride or Phosphorus Trichloride using standard procedures. The purified 3-(aminomethyl)-5-methylhexanoyl chloride was combined with 3,5-dimethyltricyclo[3.3.1.13,7]decan-1-amine (FORMULA VIII). For Example; To 500 ml. of dichloromethane taken in a 1000 ml. round bottom flask under nitrogen purge, was added 100.0 gm. of 3,5-dimethyltricyclo[3.3.1.13,7]decan-1-amine. While cooling the flask in an ice water bath, 65.4 gm. of 3-(aminomethyl)-5-methylhexanoyl chloride was added at a rate sufficient to maintain the temperature of the reaction mixture at between about 20′ C and 25′ C. The ice bath was removed and the mixture stirred for one hour. Then added to the mixture while still stirring, 6.1 gm. of thionyl chloride. The mixture continued to be stirred for one hour. Then the solvent and other volatiles were vacuumed and distilled, leaving heavy amber-colored oil. To it were added 287 mL of ethyl acetate and the mixture was stirred for two hours resulting in a fine white precipitate. These white solid particles were collected under vacuum on a Buchner funnel and washed thoroughly with ethyl acetate. Upon drying at 80′ C., a yield of 100.7 gm. of compound of formula 1 was recovered having analytical purity. The Molecular weight: 320.51, The part I was exhibited ¹HNMR (CDCl₃, 400 MHZ): δ0.833 (6H, singlet), 1.156 (2H, quartet), 1.328 (4H, quartet), 1.683 (4H, quartet), 1.869 (2H, broad signal), 2.179 (1H, broad signal), 5.28 (3H, broad signal) and the part II exhibited the following; 2.95 (IH, dd, J=12.84 Hz and 3.54 Hz), 2.82 (IH, dd, J=12.82 Hz and 7.94 Hz), 2.44 (IH, dd, J=15.73 Hz and 3.37 Hz), 2.25 (IH, dd, J=15.70 Hz and 8.76 Hz), 2.06 (IH, m), 1.69 (IH, m), 1.23 (2H, m), 0.92 (6H, t, J=6.42 Hz).

Mass Spectra; MS (Q-T of micro, ESI+): 179 (M+320.28).

Element Analysis (C₂₀H₃₆N₂₀), (Actual results) Calculated value %: C, (74.95%) 74.93%; H, (11.32%) 11.33%; N, (8.74%) 8.73%; O, (4.99%) 4.99%.

### Example 2

### Preparation of Compound of Formula 31

3-[1-(dimethylamino)-2-methylpentan-3-yl]phenol (FORMULA IX) was treated with para nitrophenyl chloroformate in triethyl amine/Dry Chloroform at 0″ C to convert into its para nitro phenoxy carbonate ester according to Scheme IV. For Example 100 mg 3-[1-(dimethylamino)-2-methylpentan-3-yl]phenol was dissolved in 5 mL of dry Chloroform under inert atmosphere in a round-bottom flask. The solution was cooled down to 0′ C and 0.05 ml of triethyl amine was added very slowly and the reaction mixture was allowed to stir for 5 minutes. Para-nitrophenyl chloroformate 50 mg was dissolved in 10 mL of dry chloroform and was added to the 3-[1-(dimethylamino)-2-methylpentan-3-yl]phenol reaction mixture slowly and the reaction mixture was then allowed to warm to room temperature. The completion of the reaction was determined by thin layer chromatography. The resulting solution was dried by removing the solvents under vacuum to obtain an oily solid material which is dissolved and dried repeatedly to remove the unwanted by products. The Para nitrophenoxy carbonate ester of 3-[1-(dimethylamino)-2-methylpentan-3-yl]phenol (Yield 22%). The resulting Para nitrophenoxy carbonate ester of 3-[1-(dimethylamino)-2-methylpentan-3-yl]phenol in turn treated with 3-(aminomethyl)-5-methylhexanoic acid (FORMULA I) in triethyl amine and THF solvent at 0′ C. For Example: 100 mg of 3-(aminomethyl)-5-methylhexanoic acid (FORMULA I) was dissolved in 5 ml tetrahydrofuran solvent under inert conditions (Nitrogen gas) and 0.05 ml of triethyl amine was added gradually over a period of time and cooled to 0′ C. Separately 100 mg of para nitrophenoxy carbonate ester of 3-[1-(dimethylamino)-2-methylpentan-3-yl]phenol was dissolved in 5 ml of dry Tetrahydrofuran solvent by constant stirring. The resulting reaction mixture was cooled to 0′ C. The two reaction mixtures were gradually mixed by constant stirring at 0′ C under inert conditions and the reaction mixture was allowed to gradually warm up room temperature. The compound of Formula 31 was extracted by standard work up and purified by chromatography (Yield 15%).

Elemental analysis: (Actual results) Calculated value %: C, (67.91%) 67.95%; H, (9.48) 9.42; N, (6.87) 6.89; and O was calculated as 15.74%.

MS (Q-T of micro, ESI+): M+: 406.2628

### Example 3

### Preparation of Compound of Formula 25

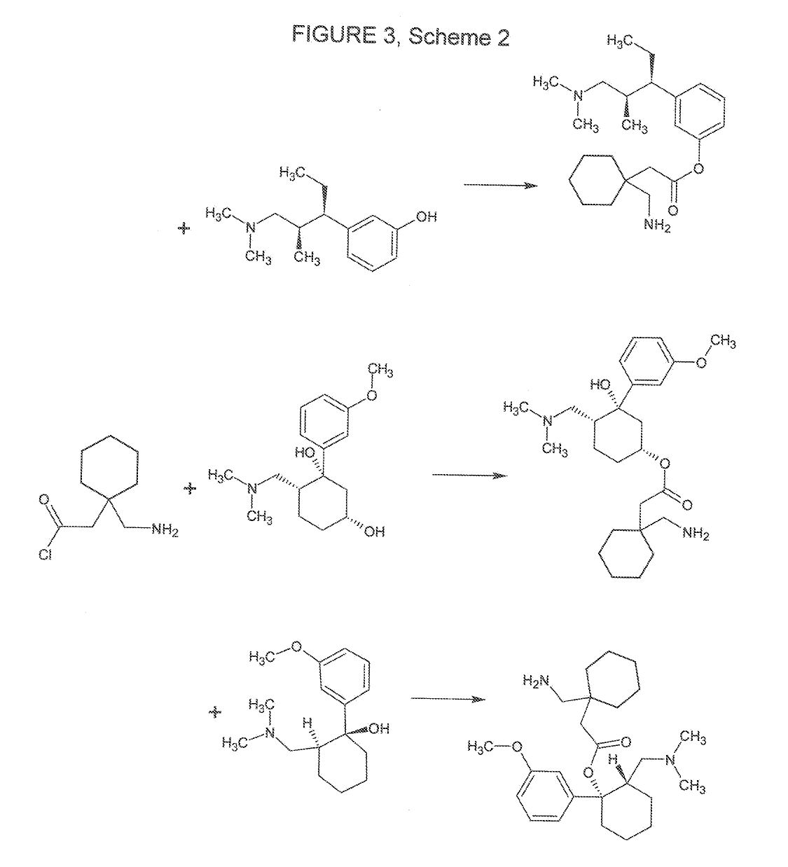

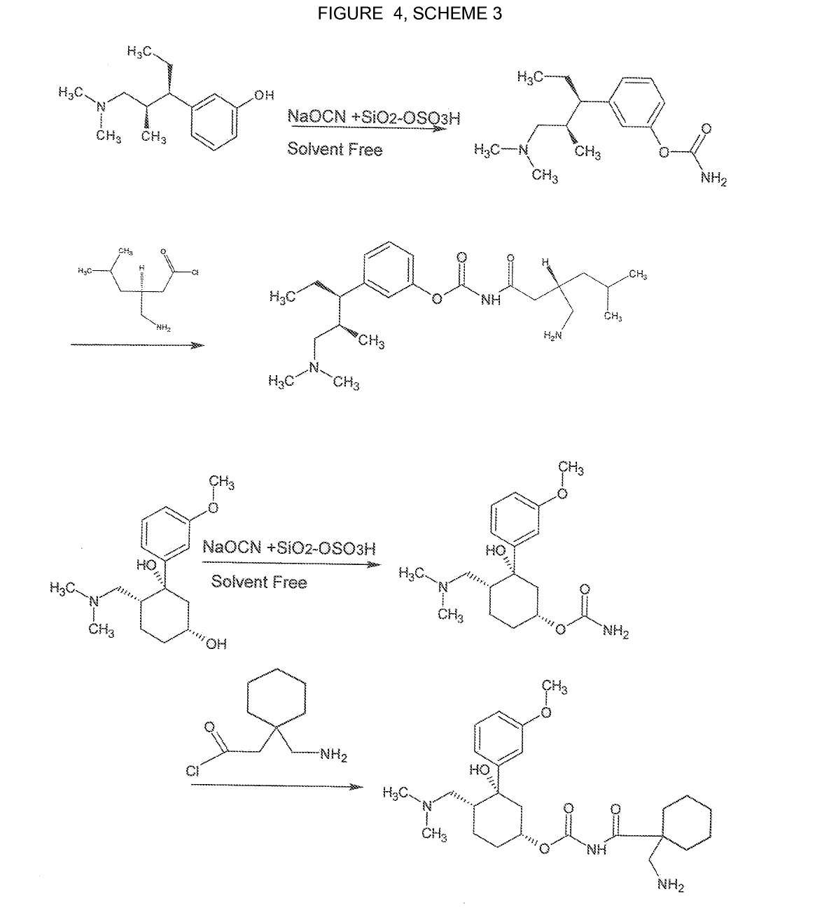

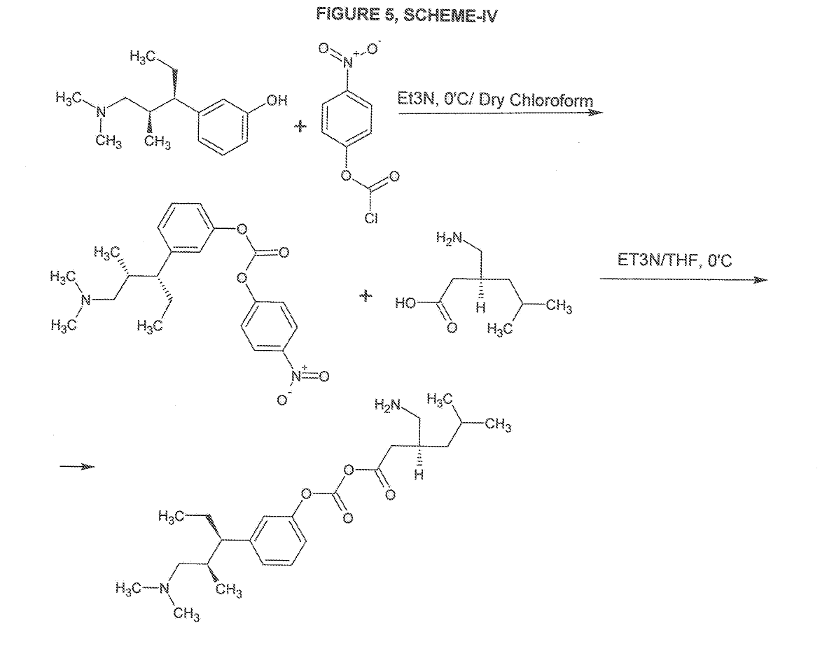

The compound of the Formula 25 can be prepared according to standard esterification procedures as outlined under Scheme II (FIG. 3).

A mixture of 1-(aminomethyl)cyclohexyl acetic acid and 3-[1-(dimethylamino)-2-methylpentan-3-yl]phenol with a small amount of sulphuric acid is preheated and fed to an esterifying column where it is refluxed. The mixture removed goes to a second refluxing column where a ternary azeotrope containing 45% of compound of Formula 25 is removed. Water is mixed with the distillate after which it separates into two layers. The top layer is fed to a refluxing column from which the residue containing 95% of compound of Formula 25 is distilled to remove any impurities.

The compound of Formula 25 having molecular formula C24H39N3O3 was analyzed. CHN analysis showed (Actual results) Calculated value %: C, (69.04) 69.02%; H, (9.4) 9.42%; N, (10.05) 10.07%; and O was calculated as (11.52) 11.50%. The compound had M⁺ (MS (Q-T of micro, ESI+) of 417.298

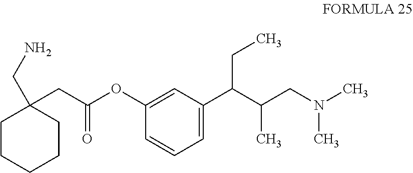

### Example 4

### Preparation of Compound of Formula 41

The compound of the Formula 41 can be prepared according to standard etherification procedures as outlined below

The starting materials 2-(bromomethyl)-4-methylpentan-1-amine and 3-[(2R,3R)-1-(dimethylamino)-2-methylpentan-3-yl]phenol were reacted together at room temperature for two days in the presence of 2 equivalent of triethyl amine, one equivalent of methyl sulfate in a solvent mixture of acetone and Perfluorohexane. With standard work up procedures and HPLC procedures yield (30%) compound of Formula 41 (Mol Weight 334.53), Elemental Analysis (Actual %) Calculated % are C, (75.41%) 75.39%; H, (11.47%) 11.45%; N, (8.39%) 8.37%; and O, 4.78%. The mass ion showed (M+): 334.2978 DA.

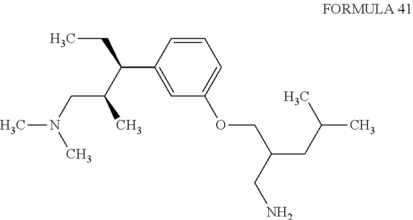

### Clinical Experiments

The compounds of this invention have been prepared using a number of starting materials outlined in Formula I to XIV.

The novel compounds of this invention are of significant therapeutic benefit and it is demonstrated in a clinical study involving tail-flick test on rats was performed according to the following procedure.

Therapeutic Solutions: The compound 290 mg was dissolved in 30 ml of saline solution and the injection volume was calculated as 1 ml/kg to achieve 3 mg/kg of compound of Formula 31. The placebo solution was identical except it is devoid of any drug.

### Tail-Flick Test:

The experimentation was started by selecting ten at least 90-100 days old and were all weighed to determine the volume of experimental and placebo solution. Each rat, was habituated for three days to handling and the tail flick procedure and without heat exposure. In the administration, each animal was only used for one day of experiments, and given only one dose of drug, or placebo solution. The technique of D'Amour and Smith (1941) relies on latency to flick of the tail from onset of a focused beam of intense light. This method, employed for decades, has now been augmented by the ability to monitor temperature at the site of exposure. Columbus Instruments model TF-2 model was warmed, on the day of the experiment, for at least 30 minutes. The intensity of the lamp was adjusted so that baseline tail-flick latency for rats is equal to approximately 2.0 seconds. The intensity was set to 40% as this was determined to be the ideal intensity from the intensity response curve. The apparatus should be programmed to use a cut-off point of 10 seconds to prevent tissue damage to the rats in the case that the tail does not flick. The experimental rat was placed in mitten and its tail was blackened for suitable length. The tail was placed in the groove and heat exposure was started. The lamp of the apparatus shuts down automatically whenever the tail flicks from the heat source. The baseline value was determined for each animal prior to injection. The TFL was measured approximately every hour. The semi-log means were calculated.

### Results

The compounds of this invention have been prepared using a number of starting materials outlined in Formula I to XIV.

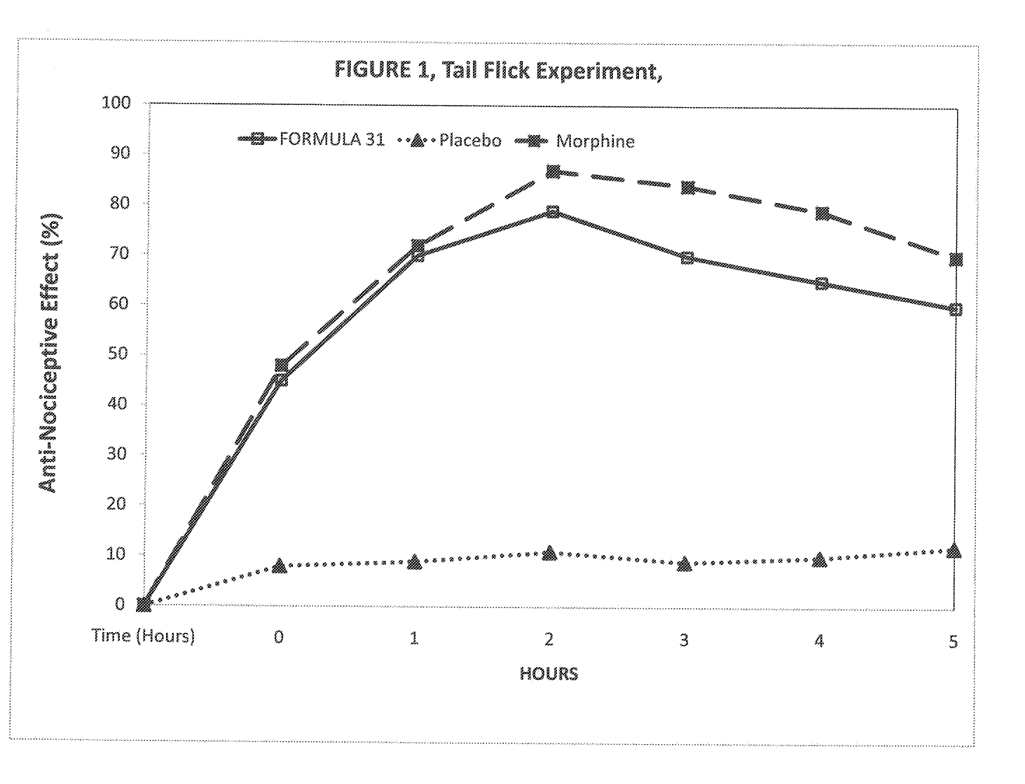

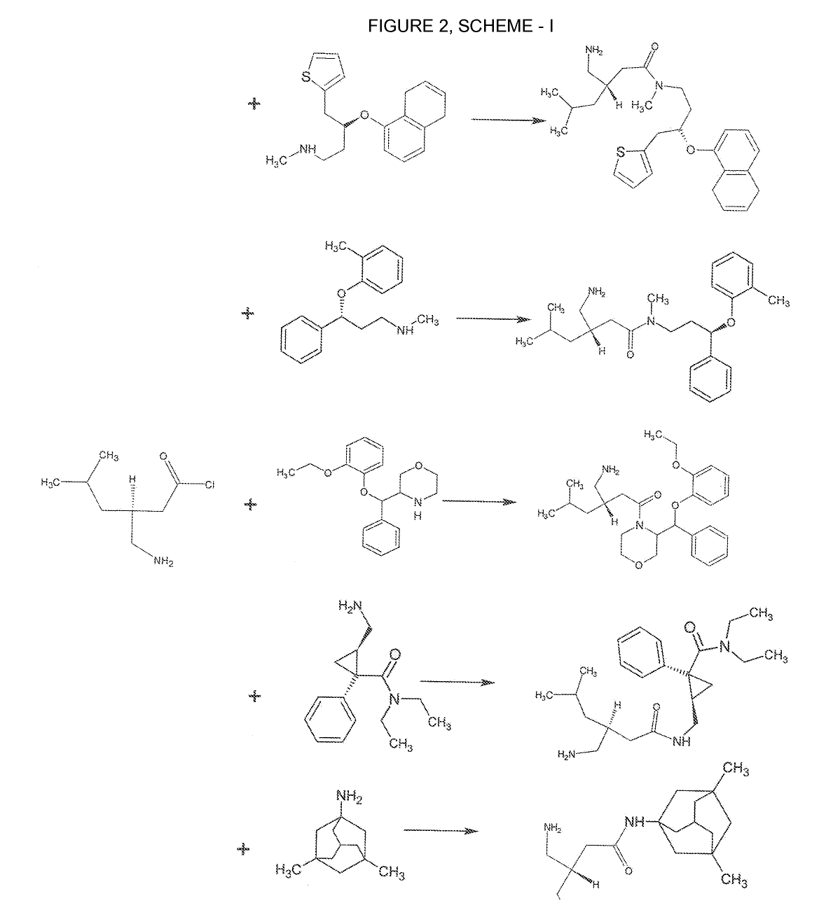

The invention discloses a number of novel therapeutic compounds that are useful for treating disorder in a mammal as demonstrated in FIG. 1.

### Hot Plate;

The device consisted of an electrically heated surface and an open plexiglass tube (34 cm high×44 cm diameter), as per the hot plate test adapted from Eddy and Leimbach (1953); The animals were confined to the heated surface. The temperature was kept at 46.0° C.±1.0° C. Mice were placed on the hot plate, and the time, recorded with a stopwatch, until either licking of the hind paw or jumping were initiated. Animals were tested prior to and 30 min after drug administration. The pre-drug latencies were between 17 and 45 s. The maximum possible effect (MPE) was defined as the lack of a nociceptive response during the exposure to the heat stimulus. The percentage of MPE was calculated according to the formula: [(T1−T0)/(T2−T0)]×100, where T0 and T1 were the latencies obtained before and after drug injection, and T2 was the cut-off time (120 s).

### Results

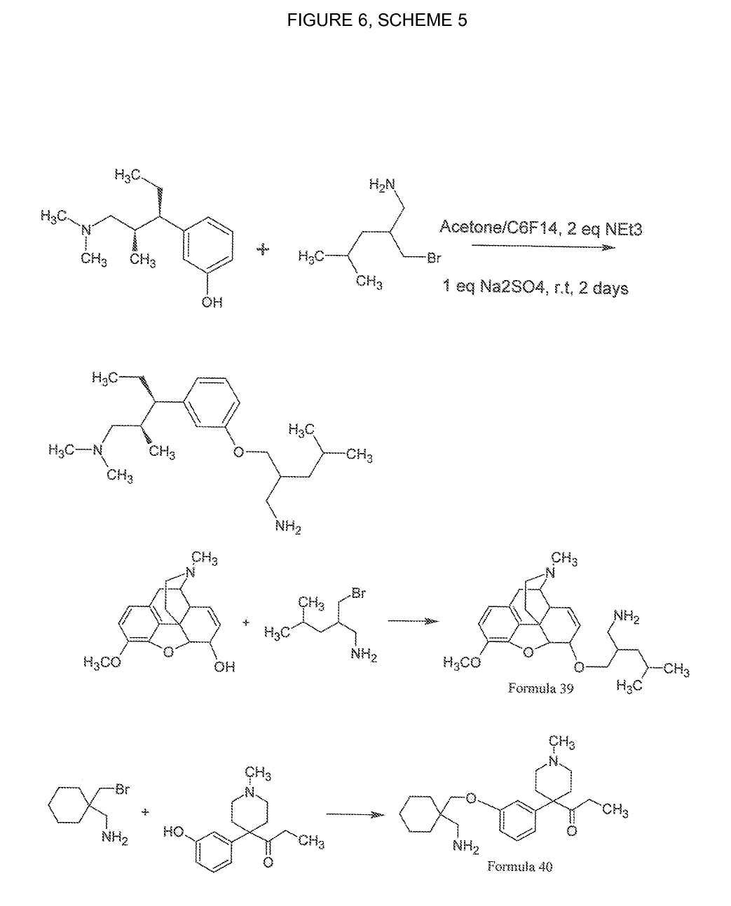

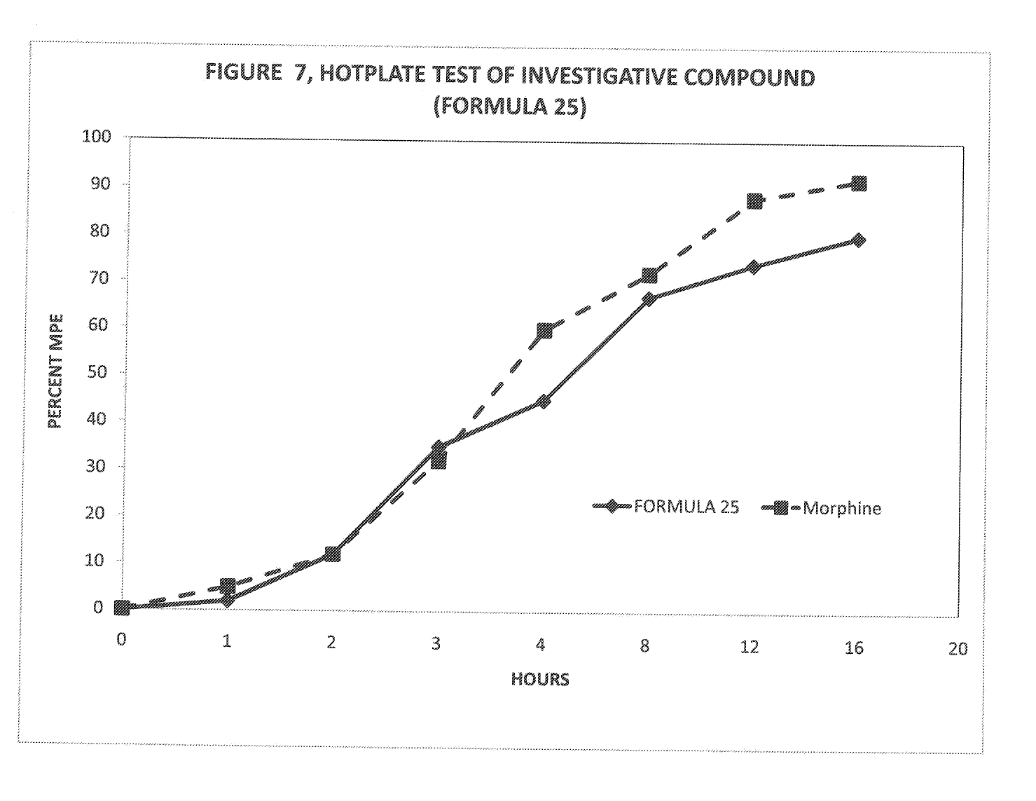

Compound of Formula 25 [F(4,44)=8.82, p<0.0001] and morphine [F(4,45)=13.35, p<0.0001] induced dose-dependent antinociception and reached full efficacy. (FIG. 6).

FIG. 7 shows Effect of investigative compound and morphine in the hot plate model of acute thermal pain in mice. Data are expressed as mean (±SEM) % MPE. p<0.05 versus vehicle control.

1. A compound of formula (I):
X—Y—Z (I)
or pharmaceutically acceptable salt thereof;
wherein,
X and Z, which may be same or different, are independently selected from substituted or unsubstituted alkyl, substituted or unsubstituted alkenyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalkyl;
Y is a linker selected from —O—, —S—, —NH—, —(CH₂)ₙ—, —CO—, —CONR^{a}—, —NR^{a}CO—, —NR^{a}COO—, —COO—, —CONR^{a}CO—, —CONR^{a}COO— and —COOCOO—;
at each occurrence, R^{a} is selected from hydrogen, substituted or unsubstituted alkyl, substituted or unsubstituted alkenyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalkyl; and
‘n’ is an integer selected from 0 to 8, both inclusive.

2. A compound of claim 1, having structural formula (Ia) 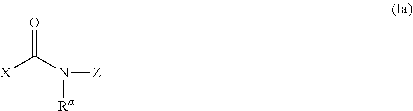
or pharmaceutically acceptable salt thereof;
wherein,
R^{a} is selected from hydrogen and substituted or unsubstituted alkyl; and
X and Z, which may be same or different, are independently selected from the group consisting of 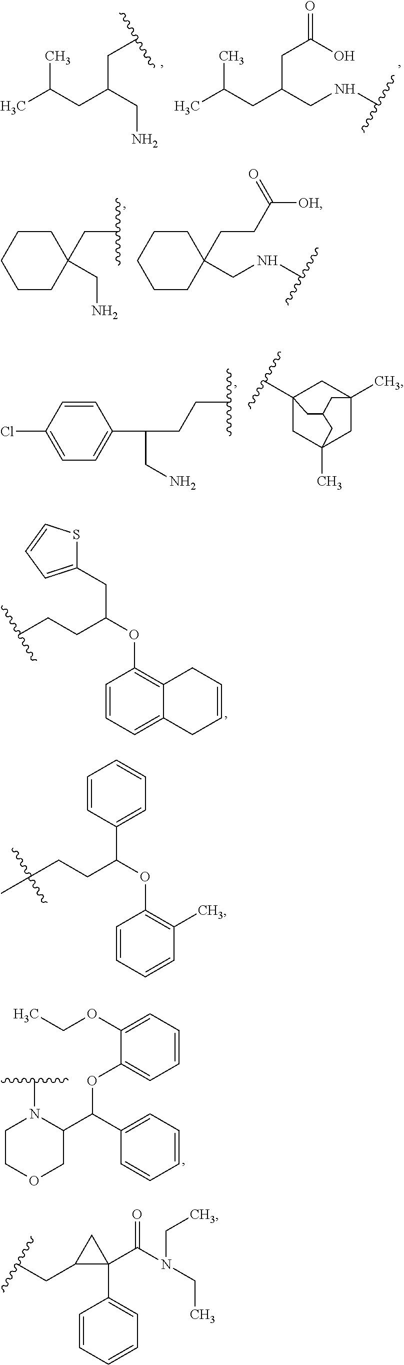 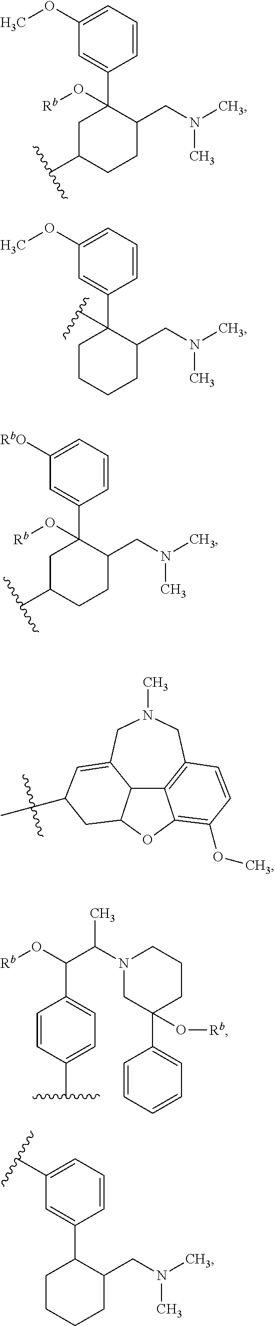 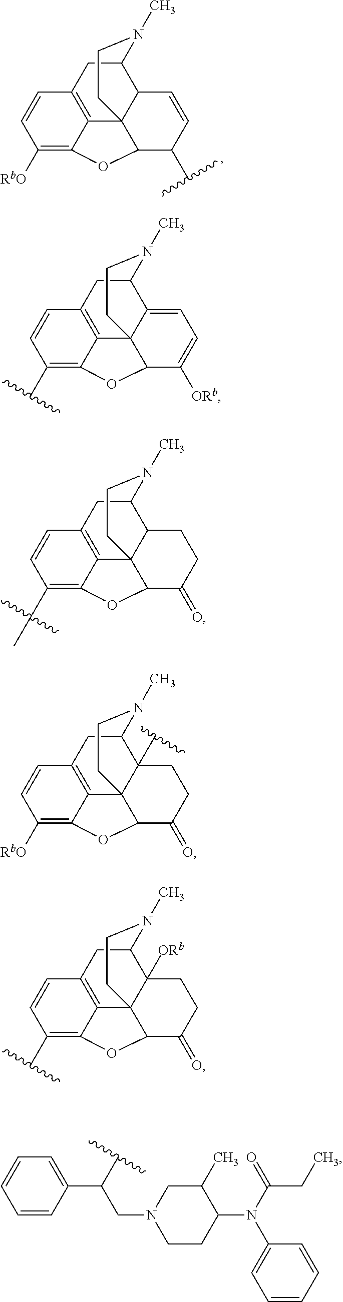 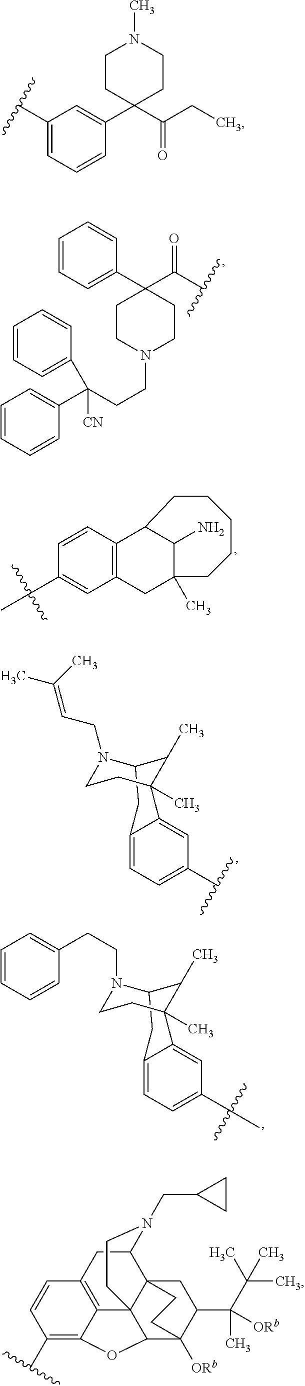 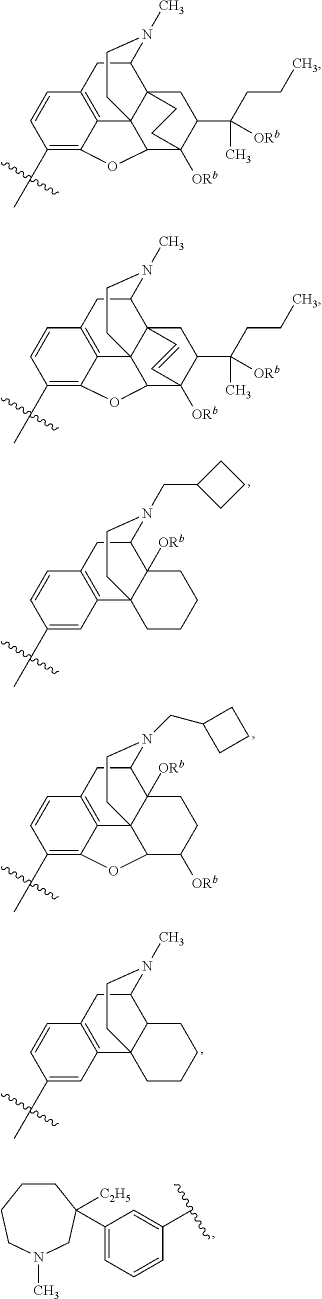 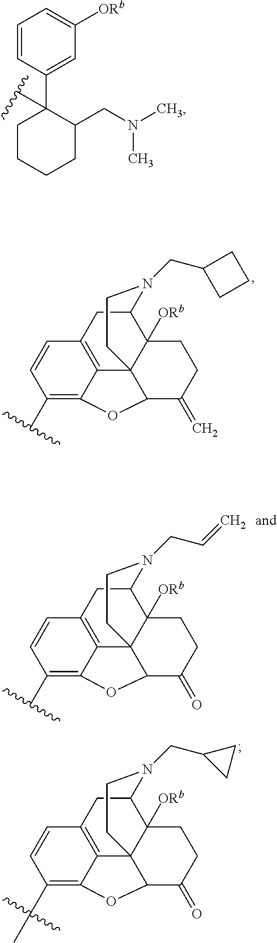
at each occurrence, R^{b} is selected from hydrogen, substituted or unsubstituted alkyl, substituted or unsubstituted alkenyl, substituted or unsubstituted cycloalkyl, substituted or unsubstituted cycloalkylalkyl, substituted or unsubstituted cycloalkenyl substituted or unsubstituted aryl, substituted or unsubstituted arylalkyl, substituted or unsubstituted heteroaryl, substituted or unsubstituted heteroarylalkyl, substituted or unsubstituted heterocyclic group or substituted or unsubstituted heterocyclylalky; or R^{b} is a protecting group;

3. The compound of claim 2, wherein R^{a} is hydrogen.

4. The compound of claim 2, wherein R^{a} is methyl.

5. The compound of claim 2, wherein R^{b} is hydrogen.

6. The compound of claim 2, wherein R^{b} is methyl.

7. The compound of claim 2, wherein R^{b} is a protecting group.

8. A compound of claim 1, having structural formula (Ib) 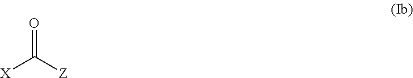
or pharmaceutically acceptable salt thereof;
wherein, X and Z are as defined above in formula (Ia).

9. A compound of claim 1, having structural formula (Ic) 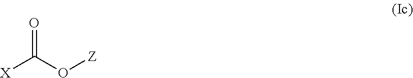
or pharmaceutically acceptable salt thereof;
wherein, X and Z are as defined above in formula (Ia).

10. A compound of claim 1, having structural formula (Id) 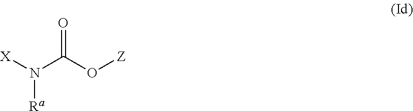
or pharmaceutically acceptable salt thereof;
wherein, X, Z and R^{a} are as defined above in formula (Ia).

11. The compound of claim 10, wherein R^{a} is hydrogen.

12. The compound of claim 10, wherein R^{a} is methyl.

13. A compound of claim 1, having structural formula (Ie) 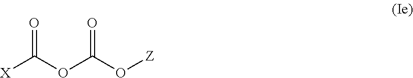
or pharmaceutically acceptable salt thereof;
wherein, X and Z are as defined above in formula (Ia)

14. A compound of claim 1, having structural formula (If) 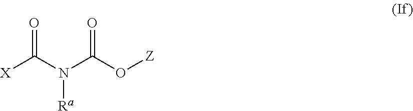
or pharmaceutically acceptable salt thereof;
wherein, X, Z and R^{a} are as defined above in formula (Ia).

15. The compound of claim 14, wherein R^{a} is hydrogen.

16. The compound of claim 14, wherein R^{a} is methyl.

17. A compound of claim 1, having structural formula (Ig) 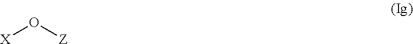
or pharmaceutically acceptable salt thereof;
wherein, X and Z are as defined above in formula (Ia).

18. The compound of claim 1, having structural Formula 1

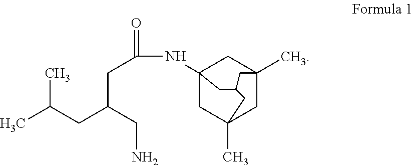

19. The compound of claim 1, having structural Formula 2

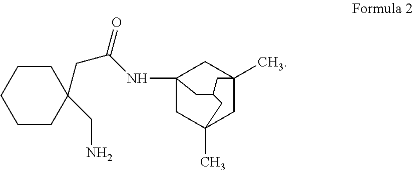

20. The compound of claim 1, having structural Formula 3

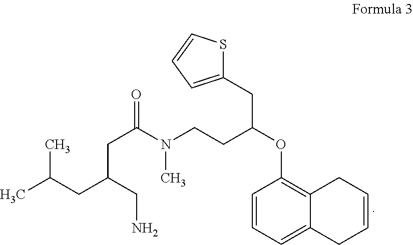

21. The compound of claim 1, having structural Formula 4

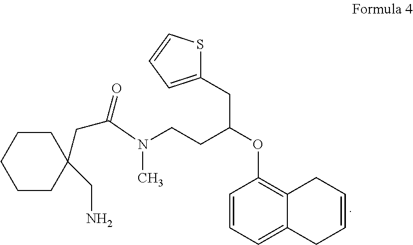

22. The compound of claim 1, having structural Formula 5

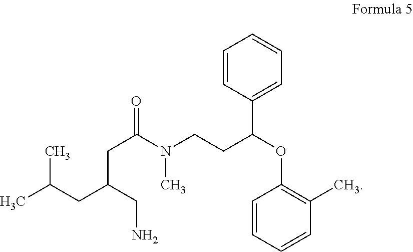

23. The compound of claim 1, having structural Formula 6

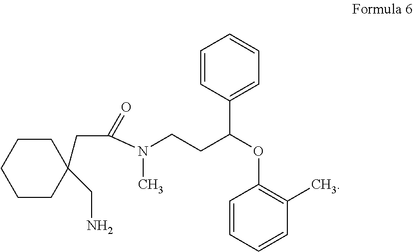

24. The compound of claim 1, having structural Formula 7

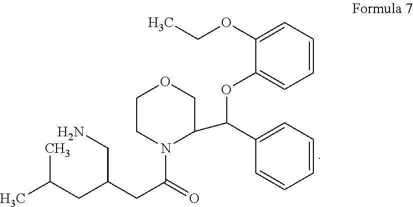

25. The compound of claim 1, having structural Formula 8

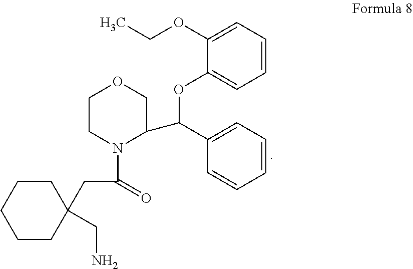

26. The compound of claim 1, having structural Formula 9

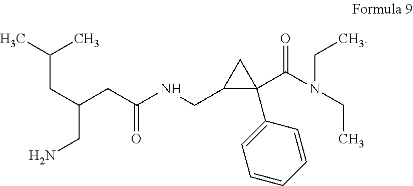

27. The compound of claim 1, having structural Formula 10

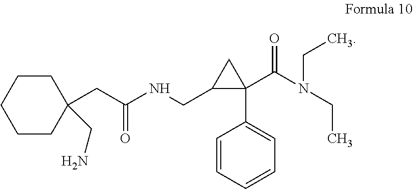

28. The compound of claim 1, having structural Formula 11

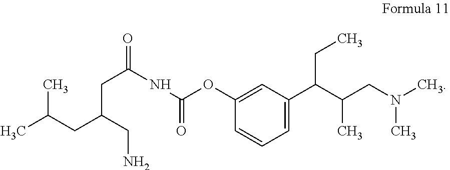

29. The compound of claim 1, having structural Formula 12

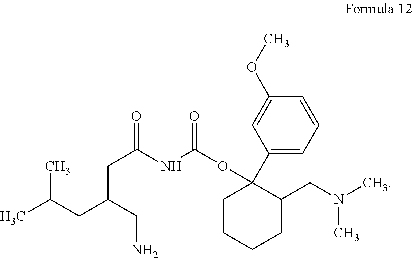

30. The compound of claim 1, having structural Formula 13

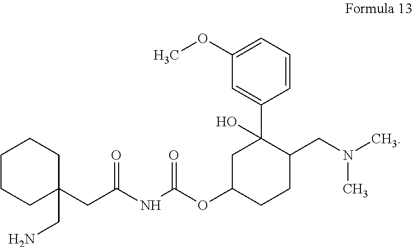

31. The compound of claim 1, having structural Formula 14

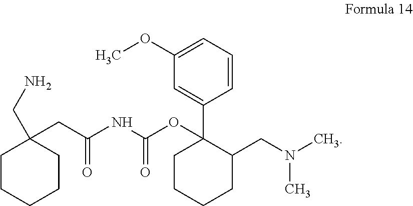

32. The compound of claim 1, having structural Formula 15

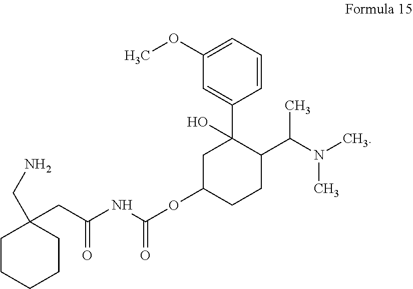

33. The compound of claim 1, having structural Formula 16

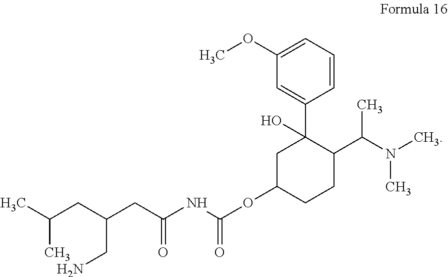

34. The compound of claim 1, having structural Formula 17

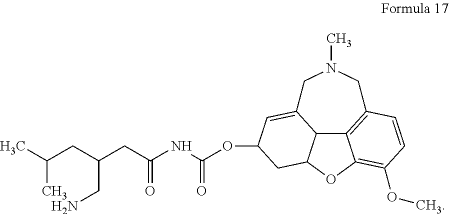

35. The compound of claim 1, having structural Formula 18

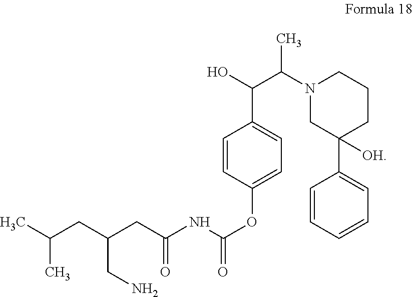

36. The compound of claim 1, having structural Formula 19

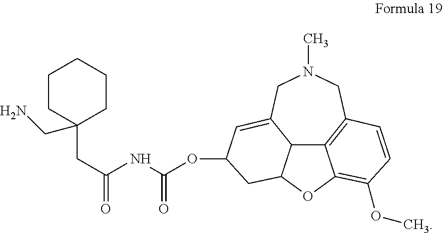

37. The compound of claim 1, having structural Formula 20

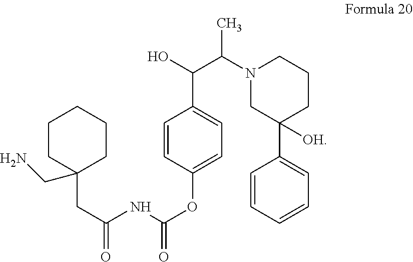

38. The compound of claim 1, having structural Formula 21

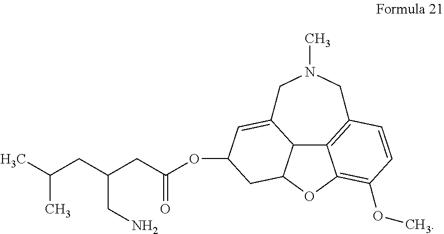

39. The compound of claim 1, having structural Formula 22

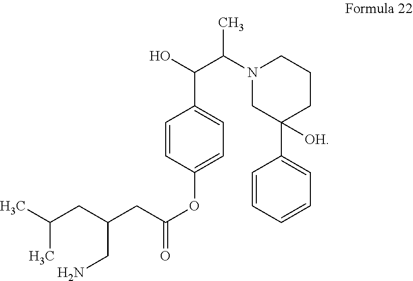

40. The compound of claim 1, having structural Formula 23

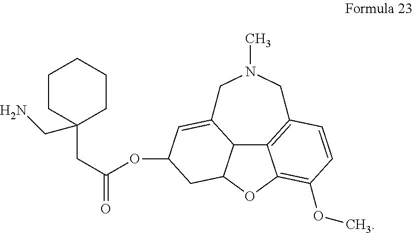

41. The compound of claim 1, having structural Formula 24

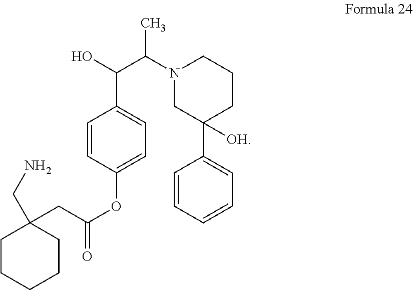

42. The compound of claim 1, having structural Formula 25

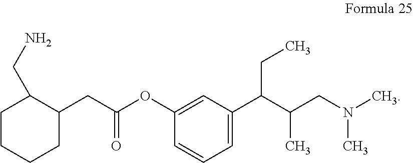

43. The compound of claim 1, having structural Formula 26

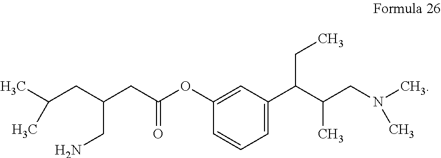

44. The compound of claim 1, having structural Formula 27

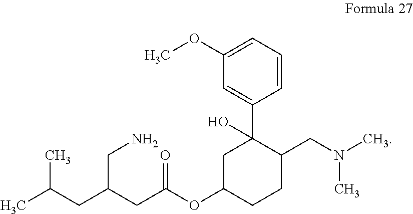

45. The compound of claim 1, having structural Formula 28

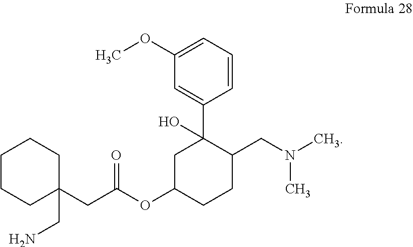

46. The compound of claim 1, having structural Formula 29

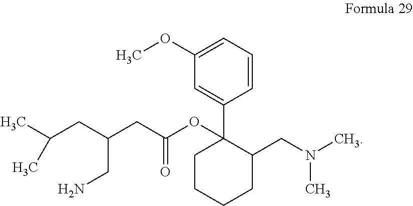

47. The compound of claim 1, having structural Formula 30

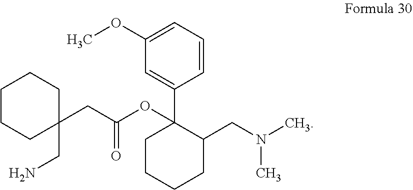

48. The compound of claim 1, having structural Formula 31

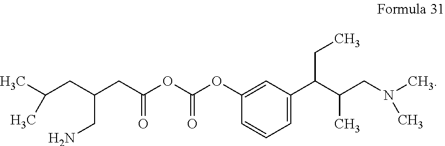

49. The compound of claim 1, having structural Formula 32

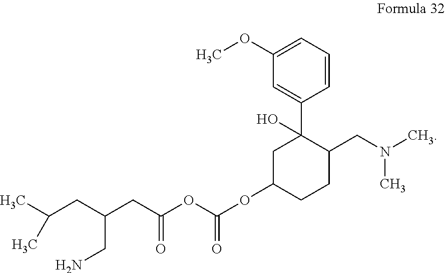

50. The compound of claim 1, having structural Formula 33

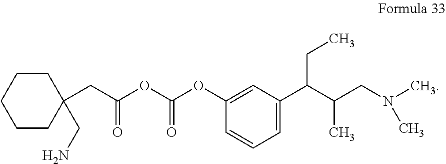

51. The compound of claim 1, having structural Formula 34

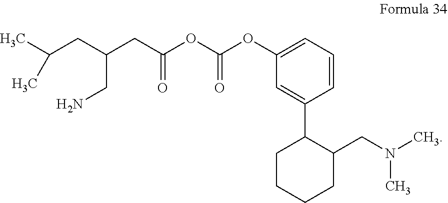

52. The compound of claim 1, having structural Formula 35

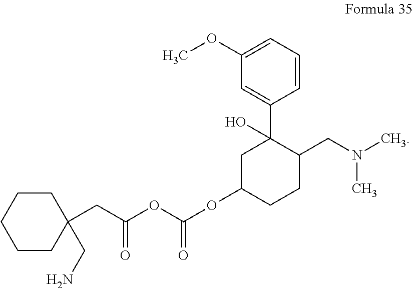

53. The compound of claim 1, having structural Formula 36

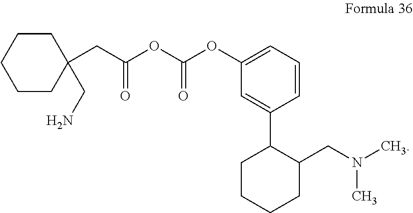

54. The compound of claim 1, having structural Formula 37

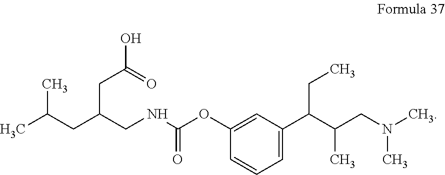

55. The compound of claim 1, having structural Formula 38

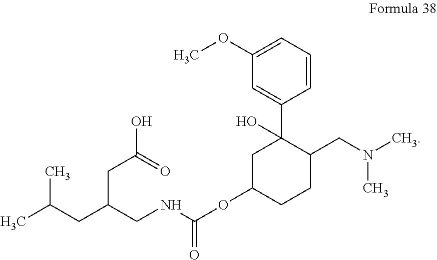

56. The compound of claim 1, having structural Formula 39

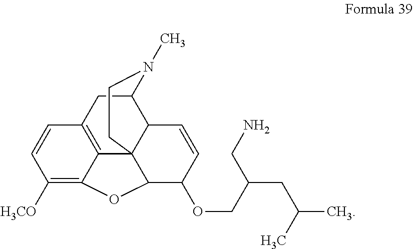

57. The compound of claim 1, having structural Formula 40

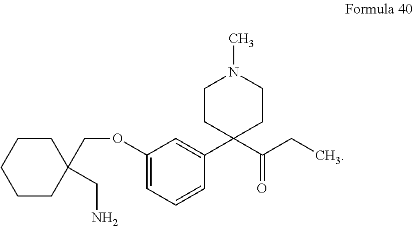

58. The compound of claim 1, having structural Formula 41,

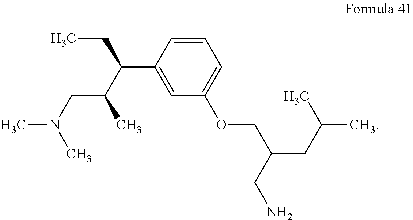

59. A pharmaceutical composition comprising a therapeutically effective amount of the compound of claim 1 or a pharmaceutically acceptable salt thereof.

60. A method of treating a disorder in a patient in need thereof, said disorder being one selected from the group consisting of neurodegenerative disorders, depression, Alzheimer's disease, cognitive disorders, motor disorders, Parkinson's disease, drug addiction, behavioral disorders, inflammatory disorders, stomach disorders, cancers, acute pain, chronic pain and recurrent pain, said method comprising administering to said patient an amount effective to treat said disorder of the compound of claim 1 or a pharmaceutically acceptable salt thereof
